(19) United States
(12) Patent Application Publication
(10) Pub. No.: US 2022/0356494 A1
(43) Pub. Date: Nov. 10, 2022

(54) METHODS AND COMPOSITIONS FOR TREATING CANCER

(71) Applicants: Engine Biosciences Pte. Ltd., Singapore (SG); Massachusetts Institute of Technology, Cambridge, MA (US)

(72) Inventors: Tackhoon Kim, Seoul (KR); Timothy Lu, San Francisco, CA (US); Stephen Harrison, El Granada, CA (US); Christine Taylor Brew, Montara, CA (US); Grace Anderson, Oakland, CA (US); Sylvain Baron, Palo Alto, CA (US); Jessie Peh, Sunnyvale, CA (US); Shawn Yost, San Mateo, CA (US); Oliver Purcell, San Francisco, CA (US); Siting Zhang, Singaporte (SG); Toni Kline, Palo Alto, CA (US)

(21) Appl. No.: 17/842,983

(22) Filed: Jun. 17, 2022

Related U.S. Application Data
(63) Continuation of application No. PCT/US2020/065335, filed on Dec. 16, 2020.
(60) Provisional application No. 62/951,479, filed on Dec. 20, 2019.
(60) Provisional application No. 63/114,930, filed on Nov. 17, 2020.

(51) Int. Cl. C12N 15/90; C12N 9/22; C12N 9/12; C07K 14/82

(52) U.S. Cl. CPC C12N 15/907; C12N 9/22; C12N 9/1205; C07K 14/82; C12Y 207/10001

(57) ABSTRACT

The present disclosure provides lethal gene pair targets for cancer treatment, along with methods and compositions for regulating their expression and activity. Gene pairs disclosed herein include tyrosine kinase genes (e.g., SRC, RON, and YES). Also provided are methods and compositions for regulating tyrosine kinase activity, including RON specific pyrazole benzamide inhibitors and methods for gene regulation.

### CROSS-REFERENCE

This application is a continuation application of International Application No. PCT/US2020/065335, filed Dec. 16, 2020, which claims the benefit of U.S. Provisional Patent Application No. 63/114,930, filed Nov. 17, 2020, and U.S. Provisional Patent Application No. 62/951,479, filed on Dec. 20, 2019, each of which is incorporated by reference herein in its entirety.

### BACKGROUND

Triple-negative breast cancer is an aggressive subtype of breast cancer and may represent about 15-20% of breast cancer occurrences. Treatment of triple-negative breast cancer is difficult due to the lack of specific target genes to which the cancer cells are sensitive.

One approach for treating cancer cells includes identifying target genes to which the cancer cells are sensitive. For example, identifying synthetic lethal gene pairs, in which an inhibition of both genes leads to cell death, may be useful therapeutically in killing cancer cells while maintaining viability of non-cancer cells.

### SUMMARY

Recognized herein is a need for improved agents for targeted treatments and therapies for cancer, and for combinations of agents that may be used to effectively target cancer cells.

In an aspect, provided herein is a method for treating a subject having or suspected of having a cancer, comprising: administering to the subject therapeutically effective amounts of one or more agents that cause a decrease in expression or activity of both members of one or more gene pairs selected from Table 1.

In some embodiments, the one or more agents comprise one or more members selected from the group consisting of a small molecule, a protein, a peptide, a ribonucleic acid (RNA) molecule, and, an endonuclease complex and a deoxyribonucleic acid (DNA) construct. In some embodiments, the DNA construct comprises an endonuclease gene. In some embodiments, the endonuclease gene encodes a CRISPR associated (Cas) protein. In some embodiments, the Cas is Cas9. In some embodiments, the DNA construct comprises a guide RNA directed to a gene of the one or more gene pairs. In some embodiments, the endonuclease complex comprises an endonuclease. In some embodiments, the endonuclease is a CRISPR associated (Cas) protein. In some embodiments, the small molecule comprises a Src inhibitor. In some embodiments, the Src inhibitor comprises N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib), N-(5-chloro-1,3-benzodioxol-4-yl)-7-(2-(4-methylpiperazin-1-yl)ethoxy)-5-(tetrahydro-2H-pyran-4-yloxy)quinazolin-4-amine (saracatinib), 4-[(2,4-dichloro-5-methoxyphenyl)amino]-6-methoxy-7-[3-(4-methylpiperazin-1-yl)propoxy]quinoline-3-carbonitrile (bosutinib), (4-Amino-5-(4-methylphenyl)-7-(t-butyl)pyrazolo[3,4-d]-pyrimidine), PP2 (4-chlorophenyl)-7-(t-butyl)pyrazolo[3,4-d]-pyrimidine) (PP1), 1-tert-butyl-3-(4-chlorophenyl)pyrazolo[3,4-d]pyrimidin-4-amine (PP2), 6-(2,6-dichlorophenyl)-2-{[3-(hydroxymethyl)phenyl]amino}-8-methyl-7H,8H-pyrido[2,3-d]pyrimidin-7-one (PD1663266), (E)-N-[4-[3-chloro-4-(pyridin-2-ylmethoxy)anilino]-3-cyano-7-ethoxyquinolin-6-yl]-4-(dimethylamino)but-2-enamide (neratinib), 3-(2-imidazo[1,2-b]pyridazin-3-ylethynyl)-4-methyl-N-[4-[(4-methylpiperazin-1-yl)methyl]-3-(trifluoromethyl)phenyl]benzamide (ponatinib), (E)-N-[4-(3-chloro-4-fluoroanilino)-3-cyano-7-ethoxyquinolin-6-yl]-4-(dimethylamino)but-2-enamide (pelitinib), N-benzyl-2-[5-[4-(2-morpholin-4-ylethoxy)phenyl]pyridin-2-yl]acetamide (Tirbanibulin), 4-methyl-3-[(2-methyl-6-pyridin-3-ylpyrazolo[3,4-d]pyrimidin-4-yl)amino]-N-[3-(trifluoromethyl)phenyl]benzamide (NVP-BHG712), (2S,3S)-2,3-dihydroxybutanedioic acid; 6-(4-methylpiperazin-1-yl)-N-(5-methyl-1H-pyrazol-3-yl)-2-[(E)-2-phenylethenyl]pyrimidin-4-amine (ENMD-2076), 4-[4-[(5-tert-butyl-2-quinolin-6-ylpyrazol-3-yl)carbamoylamino]-3-fluorophenoxy]-N-methylpyridine-2-carboxamide (Rebastinib) or any combination thereof. In some embodiments, the small molecule comprises a Yes inhibitor. In some embodiments, the Yes inhibitor comprises (3Z)-N,N-Dimethyl-2-oxo-3-(4,5,6,7-tetrahydro-1H-indol-2-ylmethylidene)-2,3-dihydro-1H-indole-5-sulfonamide) (SU-6656). In some embodiments, the small molecule comprises a Ron inhibitor. In some embodiments, the Ron inhibitor comprises N-[4-[(2-amino-3-chloro-4-pyridinyl)oxy]-3-fluorophenyl]-4-ethoxy-1-(4-fluorophenyl)-2-oxo-3-pyridinecarboxamide (BMS777607), N1′-[3-fluoro-4-[[6-methoxy-7-(3-morpholinopropoxy)-4-quinolyl]oxy]phenyl]-N1-(4-fluorophenyl)cyclopropane-1,1-dicarboxamide (Foretinib), N-(3-fluoro-4-((2-(1-methyl-1H-imidazol-4-yl)thiazolo[5,4-d]pyrimidin-7-yl)oxy)phenyl)-1-phenyl-5-(trifluoromethyl)-1H-pyrazole-4-carboxamide (ENG-009, FIG. 20), N-(3-fluoro-4-((7-(1-methyl-1H-imidazol-4-yl)-1,6-naphthyridin-4-yl)oxy)phenyl)-1-phenyl-5-(trifluoromethyl)-1H-pyrazole-4-carboxamide (ENG-015, FIG. 20), N-(3-fluoro-4-((6-(1-methyl-1H-imidazol-4-yl)-1H-indazol-3-yl)oxy)phenyl)-1-phenyl-5-(trifluoromethyl)-1H-pyrazole-4-carboxamide (ENG-018, FIG. 20), N-(3-fluoro-4-((2-(1-methyl-1H-imidazol-4-yl)thiazolo[4,5-b]pyridin-7-yl)oxy)phenyl)-1-phenyl-5-(trifluoromethyl)-1H-pyrazole-4-carboxamide (ENG-035, FIG. 20), N-(3-fluoro-4-((2-(1-methyl-1H-imidazol-4-yl)thieno[3,2-b]pyridin-7-yl)oxy)phenyl)-1-phenyl-5-(trifluoromethyl)-1H-pyrazole-4-carboxamide (ENG-006, FIG. 21), 1-(2-(2,6-difluorophenyl)-4-oxothiazolidin-3-yl)-3-(4-((7-(3-(4-ethylpiperazin-1-yl)propoxy)-6-methoxyquinolin-4-yl)oxy)-3,5-difluorophenyl)urea (ENG-013, FIG. 21), N-[4-[(2-amino-3-chloro-4-pyridinyl)oxy]-3-fluorophenyl]-4-ethoxy-1-(4-fluorophenyl)-2-oxo-3-pyridinecarboxamide (ENG-007, FIG. 21), or any combination thereof. In some embodiments, the cancer is breast cancer. In some embodiments, the breast cancer is triple-negative breast cancer.

In another aspect, disclosed herein is a composition for treating a subject having or suspected of having a cancer, comprising a formulation comprising at least one agent present in an amount that is effective to cause a decrease in expression or activity of one or more gene pairs selected from Table 1. In some embodiments, the at least one agent comprises one or more members selected from the group consisting of a small molecule, a protein, a peptide, a ribonucleic acid (RNA) molecule, and, an endonuclease complex and a deoxyribonucleic acid (DNA) construct. In some embodiments, the DNA construct comprises an endonuclease gene. In some embodiments, the endonuclease gene encodes a CRISPR associated (Cas) protein. In some embodiments, the Cas is Cas9. In some embodiments, the DNA construct comprises a guide RNA directed to a gene of the one or more gene pairs. In some embodiments, the endonuclease complex comprises an endonuclease. In some embodiments, the endonuclease is a CRISPR associated (Cas) protein. In some embodiments, the small molecule comprises a SRC inhibitor. In some embodiments, the SRC inhibitor comprises N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib). In some embodiments, the small molecule comprises a Yes inhibitor. In some embodiments, the Yes inhibitor comprises (3Z)-N,N-Dimethyl-2-oxo-3-(4,5,6,7-tetrahydro-1H-indol-2-ylmethylidene)-2,3-dihydro-1H-indole-5-sulfonamide (SU-6656). In some embodiments, the small molecule comprises a Ron inhibitor. In some embodiments, the Ron inhibitor comprises N-[4-[(2-amino-3-chloro-4-pyridinyl)oxy]-3-fluorophenyl]-4-ethoxy-1-(4-fluorophenyl)-2-oxo-3-pyridinecarboxamide (BMS777607), or N1′-[3-fluoro-4-[[6-methoxy-7-(3-morpholinopropoxy)-4-quinolyl]oxy]phenyl]-N1-(4-fluorophenyl)cyclopropane-1,1-dicarboxamide (Foretinib). In some embodiments, the cancer is breast cancer. In some embodiments, the breast cancer is triple-negative breast cancer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically shows a workflow for decreasing expression of a gene pair using CRISPR-based mutagenesis.

FIGS. 2A-2B show example data showing the impact of dual mutation of SRC and YES on cell viability in a CRISPR-based screen. FIG. 2A illustrates bar plots of cell viability and FIG. 2B illustrates a violin plot of cell viability.

FIG. 3 shows additional example data showing the impact of dual inhibition of SRC and YES expression on cell viability in a CRISPR-based validation assay.

FIG. 4 shows additional example data of a ribonucleic acid (RNA) approach to decrease expression of Src and Yes.

FIG. 5 shows additional example data of a small molecule approach to decrease activity of Src and Yes.

FIG. 6 schematically the interaction between SRC and YES gene product associated signaling pathways.

FIG. 7 shows a table of a number of genes that are regulated by SRC, YES, or both genes.

FIG. 8 illustrates a table of instances of mutations of genes of by SRC, YES, or both genes in different tumor types.

FIG. 9 illustrates a table of the extent of co-expression of SRC and YES genes in breast cancer cells.

FIG. 10 shows example data of sensitivity of cells to inhibitors of Src and Yes.

FIGS. 11A-11B show example data of the impact of CRISPR-based mutagenesis of the SRC and RON genes on cell viability. FIG. 11A illustrates bar plots of cell viability and FIG. 11B illustrates a violin plot of cell viability.

FIGS. 12A-12C shows the impact of the sensitivity of various cancer cell lines to the inhibition of Ron activity in the presence or absence of Src inhibition. FIG. 12A shows a plot of the sensitivity of the cell lines to the inhibition of Ron activity using BMS777607. FIG. 12B shows a plot of the sensitivity of the cell lines to the inhibition of Ron using Foretinib and FIG. 12C shows a plot of the sensitivity of the cell lines to the inhibition of Ron using a Met/Ron inhibitor.

FIG. 13 schematically illustrates the interaction between SRC and RON gene-product associated signaling pathways.

FIG. 14 schematically shows a subset of the signaling pathways for p85 (PI3K pathway) that are influenced by Src and Ron function.

FIGS. 15A-15B show co-expression and co-activation of the SRC and RON gene pair in cancer types. FIG. 15A shows a plot of correlation coefficients. FIG. 15B shows a plot of co-activation.

FIGS. 16A-16B schematically show signaling pathways for SRC and RON. FIG. 16A shows a signaling pathway of SRC and RON. FIG. 16B schematically shows how dual inhibition of SRC and RON may be effective in suppressing tumor growth.

FIGS. 17A-17B show example data of RON and SRC expression in tumor tissue. FIG. 17A shows a plot of RON expression versus SRC expression in normal and cancerous tissues. FIG. 17B shows a table of expression levels of SRC and RON in triple-negative breast cancer cells.

FIG. 18 shows example in vivo data of tumor volume as a function of treatment with SRC inhibitor, a RON inhibitor or a combination of both.

FIG. 19 shows example data of a compound that can selectively inhibit RON.

FIG. 20 shows example chemical compositions of compounds that may inhibit RON.

FIG. 21 shows additional example chemical compositions of compounds that may inhibit RON.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “subject,” as used herein, generally refers to an animal, such as a mammal (e.g., human), reptile, or avian (e.g., bird), or other organism, such as a plant. For example, the subject can be a vertebrate, a mammal, a rodent (e.g., a mouse), a primate, a simian or a human. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient.

The term “genome,” as used herein, generally refers to genomic information from a subject, which may be, for example, at least a portion or an entirety of a subject's hereditary information. A genome can be encoded in a deoxyribonucleic acid (DNA) molecule (s) and may be expressed in a ribonucleic acid (RNA) molecule(s). A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

### Methods for Treating Cancer

In an aspect, provided herein are therapies (e.g., combination therapies) for the treatment of pathologies, such as cancer. A method for treating a subject having or suspected of having a cancer can comprise administering to the subject a therapeutically effective amount(s) of one or more agents that cause a decrease in expression or activity of both members of one or more gene pairs. Such one or more gene pairs may be associated with the cancer. In some examples, the one or more genes pairs are provided in Table 1. In certain instances, decreasing the activity or expression of a single member of one or more gene pairs may have little effect on cell viability, but decreasing the activity or expression of both members of one or more gene pairs results in cell death.

The one or more gene pairs may comprise a pair of genes that may be expressed in a cancer cell of the subject. In some cases, one or both members (e.g., genes) of a gene pair may be expressed in a cancer cell at a normal level (e.g., not over-expressed or under-expressed compared to a non-cancer cell of the subject), while in other cases, one or both members of a gene pair may be highly expressed in a cancer cell, or lowly expressed in a cancer cell, e.g., when compared to a control cell or population of cells. In some cases, one member of a gene pair may be expressed at a normal level, while the other member of the gene pair may be expressed at a lower or higher level than normal.

The one or more gene pairs may comprise a synthetic lethal gene pair—the inhibition or decreased expression or activity of one of the members of the gene pair alone is not sufficient to kill the cell (e.g., cancer cell), but the combination of inhibition or decreased expression of both members of the gene pair leads to cell death. In some cases, inhibition or decreased expression or activity of each of the members of the gene pair alone result in a reduction in viability of a cell or cell population, but the decreased expression or activity of both members of the gene pair results in a greater reduction in viability of the cell or cell population. For example, the decrease of expression or activity of both members may act synergistically, with a greater reduction in viability than the sum of the reductions of viability from decreased expression or activity of each member of the gene pair.

The one or more gene pairs may comprise a constitutively active gene (e.g., housekeeping gene), or a gene that is expressed independently of an external factor (e.g., ligand). In some cases, one or both members of the gene pair may encode for a protein. The protein may be an enzyme, e.g., a kinase, which may phosphorylate a substrate (e.g., another protein or ligand, e.g., lipid or carbohydrate), or transfer a phosphate group to a substrate. For example, one or both members of the gene pair may encode for a tyrosine kinase.

In some cases, one member of a gene pair may interact with the other member of the gene pair. In some cases, the one member of a gene pair may interact directly with the other member of the gene pair. For example, one of the members of the gene pair may be or encode a protein that is an upstream agonist or antagonist of the other member of the gene pair. In such an example, the upstream agonist may activate or deactivate the other member of the gene pair, e.g., in the case of a kinase, via phosphorylation of the other member of the gene pair. In some cases, the one member of a gene pair may interact indirectly with the other member of the gene pair. For example, the one member of the gene pair may be or encode a protein or enzyme that is an upstream agonist or antagonist of a protein within a protein signaling cascade or signal transduction pathway. In one such example, one of the members of the gene pair may be an agonist or antagonist of another gene (or encoded protein) that regulates the other member of the gene pair. Similarly, one of the members of the gene pair may be an agonist or antagonist of another gene (or encoded protein) that regulates yet another gene (or encoded protein) that may regulate the other member of the gene pair. In some cases, one of the members may regulate another gene or protein that is at least 1, 2, 3, 4, 5, 6, 7, 8, or more components (e.g., nodes or other genes, proteins, or signal transducers) upstream of the other member of the gene pair.

In some cases, the members of the gene pair may regulate one another, e.g., via a feedback mechanism. For example, an increase in expression of one of the members of the gene pair may increase, decrease, or otherwise change the expression level of the other member of the gene pair, and similarly, an increase in the expression of the other member of the gene pair may increase, decrease, or otherwise change the expression level of the first member of the gene pair. Each member of the gene pair may interact directly with the other member of the gene pair (e.g., may be directly upstream or downstream of the other member of the gene pair). Alternatively, the members of the gene pair may interact indirectly. For example, one of the members of the gene pair may be an agonist or antagonist of another gene (or encoded protein) that regulates the other member of the gene pair, and vice-versa. Similarly, one of the members of the gene pair may be an agonist or antagonist of another gene (or encoded protein) that regulates yet another gene (or encoded protein) that may regulate the other member of the gene pair. In some cases, one of the members may regulate another gene or protein that is 1, 2, 3, 4, 5, 6, 7, 8, or more components (e.g., nodes or other genes, proteins, or signal transducers) upstream of the other member of the gene pair.

In some instances, the members of the gene pair may regulate a subset of the same genes downstream. For example, one member of the gene pair may regulate a plurality of downstream genes, a subset of which are also regulated by the other member of the gene pair. In cases where the cell is a cancer cell, the downstream genes may comprise genes important in cancer-related processes, e.g, HIPPO pathway, epithelial-to-mesenchymal transition, P13K pathway, DNA replication, cell migration, cell metastasis, etc. Alternatively or in addition to, the members of the gene pair may be regulated by a subset of the same genes. In such cases, one member of the gene pair may be regulated by one or more upstream genes, which upstream gene or genes may also regulate the other member of the gene pair.

In cases where one of the members of the gene pair interacts with the other member of the gene pair, a gene interaction score may be used to determine how interactive the one member is with the other member, or how interactive both members of the gene pair are with one another. The gene interaction score can be calculated using a Bliss or GI score. In such cases, a list of all combinations of gene pairs in a study (e.g., all pairs of tyrosine kinase genes) may be generated. Each gene pair may then be designated a GI score, which may correspond to whether a synergistic interaction may occur. For instance, a synergistic interaction may comprise a combination of genes (e.g., two or more genes) that exhibits a stronger phenotype than predicted by the additive effect of the individual phenotypes.

The GI score may be calculated using the equation: GI_{AB}=Z_{obs}−Zₑₓₚ=Z_{AB}−(Z_{A}+Z_{B}), where A refers to gene A (one member of a gene pair), B refers to gene B (another member of a gene pair), Z_{obs} is the observed phenotype, Zₑₓₚ is the expected phenotype, and Z_{AB} is the observed phenotype. The GI score may be determined using data (e.g., publicly available data) or experimentally. GI scores may also be calculated using the approach of K. Han, E. E. Jeng, and co-authors (“Synergistic drug combinations for cancer identified in a CRISPR screen for pairwise genetic interactions” Nature Biotechnology 2017 May: 35(5): 463-474), which is incorporated by reference herein in its entirety for all purposes.

In some cases, the one or more agents used to cause a decrease in expression or activity of one or both members of the gene pair may comprise a small molecule, a protein, a peptide, a ribonucleic acid (RNA) molecule, a deoxyribonucleic acid (DNA) construct, or a combination thereof (e.g., a protein-nucleic acid complex). In an example, the one or more agents may comprise a protein-nucleic acid complex, e.g., an endonuclease complex and a DNA construct. In some cases, the endonuclease complex comprises a clustered regularly interspaced short palindromic repeat (CRISPR) associated (Cas) protein or variant thereof (e.g., an engineered variant). In such cases, the DNA construct may be co-administered with the endonuclease complex. Alternatively or in addition to, the DNA construct may comprise an endonuclease gene. In such instances, the DNA construct may comprise a gene encoding for a Cas protein or variant thereof (e.g., an engineered variant). After the DNA construct is introduced or delivered to a cell (e.g., cancer cell), the DNA construct may be transcribed and translated by the cell using the cell's own machinery (e.g., polymerases, ribosomes, etc.).

In some cases, the DNA construct may comprise a guide RNA (gRNA) sequence, which may be used to direct a protein (e.g., Cas protein) to one or both of the members of at least one gene pair. The DNA construct may comprise at least one gRNA sequence, each of which may direct the protein (e.g., Cas protein) to a different gene. The DNA construct may comprise a RNA sequence, a DNA sequence, or a combination thereof. In some cases, the DNA construct comprises: (i) a first gRNA sequence, which may be used to direct an endonuclease (e.g., Cas protein) to a targeted location or gene locus for one member of the gene pair, (ii) a second gRNA sequence, which may be used to direct an endonuclease (e.g., Cas protein) to another targeted location or gene locus for the other member of the gene pair, (iii) a first sequence (e.g., a DNA sequence) corresponding to one member of the gene pair (e.g., a gene replacement), and (iv) a second sequence (e.g., DNA sequence) corresponding to the other member of the gene pair (e.g., a gene replacement). It will be appreciated, that different combinations of RNA sequences and DNA sequences may be used in the DNA construct. Moreover, other functional sequences may be included in the DNA sequence, including, but not limited to, a barcode sequence, a tag, or other identifying sequence, a primer sequence, a restriction site, a transposition site, etc.

The endonuclease complex may comprise an endonuclease, e.g., a Cas protein, or other nucleic acid-interacting enzyme (e.g., ligase, helicase, reverse transcriptase, transcriptase, polymerase, etc.). The Cas protein may comprise any Cas type (e.g., Cas I, Cas IA, Cas IB, Cas IC, Cas ID, Cas IE, Cas IF, Cas IU, Cas III, Cas IIIA, Cas IIIB, Cas IIIC, Cas IIID, Cas IV, Cas IVA, Cas IVB, Cas II, Cas IIA, Cas IIB, Cas IIC, Cas V, Cas VI). In some instances, the Cas protein may comprise other proteins (e.g., a fusion protein) and may comprise an additional enzyme that may associate with a nucleic acid molecule (e.g., ligase, transcriptase, transposase, nuclease, endonuclease, reverse transcriptase, polymerase, helicase, etc.). The endonuclease complex may be delivered exogenously or may be encoded in the DNA construct for transcription and translation within the cell.

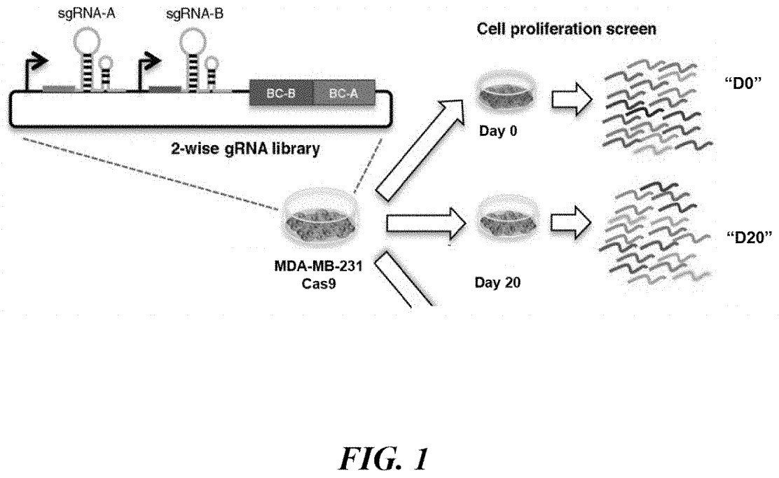

FIG. 1 schematically illustrates an example workflow for determining the effect of treatment of a population of cultured cancer cells with a protein and nucleic acid molecule. In such an example, the nucleic acid molecule can comprise a DNA construct, which may comprise a first gRNA sequence (sgRNA-A), a second gRNA sequence (sgRNA-B), a first DNA sequence (BC-B) and a second DNA sequence (BC-A). The first DNA sequence or the second DNA sequence, or both the first and the second DNA sequences may comprise a barcode sequence. The first guide sequence may have sequence homology to one of the members of the gene pair and thus may target the member of the gene pair for mutagenesis by a protein (e.g., an endonuclease, e.g., Cas9), and the second guide sequence may have sequence homology to the other member of the gene pair and thus may targets the other member of the gene pair for mutagenesis by a protein (e.g., an endonuclease, e.g., Cas9). Cells (e.g., MDA-MB-231 cells) may be treated with a therapeutically effective amount of the DNA construct and a protein (e.g., Cas9). In some cases, the DNA construct may be introduced via transfection (e.g., using a liposome or other nanoparticle) or transduction (e.g., using a virus). The protein may be administered using a nanoparticle or other vesicle, or by adding the protein to the cell culture media. The protein (e.g., Cas9) may use the sgRNA-A and sgRNA-B to direct the protein to a specific locus or location in the cell genome (e.g., at a locus of each of the members of the gene pair). Next, the protein may excise and/or replace the endogenous genes (e.g., the members of the gene pair). If replacing the endogenous genes, the protein (e.g., Cas9) may replace the endogenous genes with the first DNA sequence (BC-B) and the second DNA sequence (BC-A). Cells may then be cultivated for a duration of time (e.g., 7 days, 14 days, 20 days, etc.). The proliferation or viability of the cells may be measured, and in some instances, compared to a control population of cells. In some instances, the genome of a cell or a population of cells may be sequenced to determine if a cell or population of cells were mutated (e.g., by identification of the presence of a barcode comprised in the replacement genes, e.g., via a polymerase chain reaction (PCR) or sequencing approach).

The workflow presented in FIG. 1 may be performed in a high-throughput format. For instance, the workflow may be scaled such that 10, 50, 100, 500, 1000, 5000, 10000 or more screens may be performed, sequentially or in parallel. In such cases, each of the nucleic acid molecules or DNA constructs may comprise different sgRNA sequences. The results of such a high-throughput screening process may be used in identifying targets that may form synthetic lethal pairs (e.g., those shown in Table 1). For instance, gene pairs that show diminished cell growth, proliferation, or viability may be selected for further validation studies for synthetic lethality. In some instances, the barcode sequences may be used for identification of the gene pairs of DNA constructs present in individual cells.

In some cases, the one or more agents used to cause a decrease in expression or activity of one or both members of a gene pair may comprise a protein or peptide. For example, the one or more agents may comprise an antibody, an antibody fragment, a hormone, a ligand, or an immunoglobulin. The protein or peptide may be naturally occurring or may be synthetic. The protein may be an engineered variant of a protein (e.g., recombinant protein), or fragment thereof. The protein may be subjected to other modifications, e.g., post-translational modifications, including but not limited to: glycosylation, acylation, prenylation, lipoylation, alkylation, amidation, acetylation, methylation, formylation, butyrylation, carboxylation, phosphorylation, malonylation, hydroxylation, iodination, propionylation, S-nitrosylation, S-glutationylation, succinylation, sulfation, glycation, carbamylation, carbonylation, biotinylation, carbamylation, oxidation, pegylation, sumoylation, ubiquitination, ubiquitylation, racemization, etc. One or more modifications may be made to the protein or peptide.

In some cases, the one or more agents used to cause a decrease in expression or activity of one or both members of a gene pair may comprise a small molecule. The small molecule may be configured to decrease the expression level or activity level of one or both members of a gene pair. In some cases, the small molecule may directly interact with one or both members of the gene pair. For example, the small molecule may inhibit the protein or proteins encoded by one or both members of the gene pair, respectively. Alternatively or in addition to, the small molecule may inhibit an upstream effector or downstream protein in a signaling pathway in which one or both members of the gene pair interact.

In some cases, the small molecule inhibitor may comprise a Src inhibitor, e.g., N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib), 2-(3,4-dihydroxyphenyl)-3,5,7-trihydroxy-4H-chromen-4-one (Quercetin), PP1 or PP2 kinase inhibitor, N-(5-chloro-1,3-benzodioxo1-4-yl)-7-[2-(4-methyl-1-piperazinyl)ethoxy]-5-[(tetrahydro-2H-pyran-4-yl)oxy]-4-quinazolinamine (Saracatinib), 4-[(2,4-dichloro-5-methoxyphenyl)amino]methoxy-7-[3-(4-methylpiperazin-1-yl)propoxy]quinoline-3-carbonitrile (Bosutinib), or N-Benzyl-2-(5-(4-(2-morpholinoethoxy)phenyl)pyridin-2-yl)acetamide (KX2-391). In some cases, the small molecule inhibitor may comprise a Yes inhibitor, e.g., (3Z)-N,N-Dimethyl-2-oxo-3-(4,5,6,7-tetrahydro-1H-indol-2-ylmethylidene)-2,3-dihydro-1H-indole-5-sulfonamide (SU-6656), 6-(2,6-dichlorophenyl)-8-methyl-2-{[3(methylthio)phenyl]amino}pyrido[2,3-d]pyrimidin-7(8H)-one (PD173955), 2-{[(1R,2S)-2 aminocyclohexyl]amino}-4-{[3-(1,2,3-triazol-2-yl)phenyl]amino}pyrimidine-5-carboxamide (PRT062607), or Saracatinib. In some cases, the small molecule inhibitor may comprise a Ron inhibitor, e.g., (N-[4-(2-amino-3-chloropyridin-4-yl)oxy-3-fluorophenyl]-4-ethoxy-1-(4-fluorophenyl)-2-oxopyridine-3-carboxamide) (BMS777607), N1′-[3-fluoro-4-[[6-methoxy-7-(3-morpholinopropoxy)-4-quinolyl]oxy]phenyl]-N1-(4-fluorophenyl)cyclopropane-1,1-dicarboxamide (Foretinib), (2R)-1-[[5-[(Z)-[5-[[(2,6-Dichlorophenyl)methyl]sulfonyl]-1,2-dihydro-2-oxo-3H-indol-3-ylidene]methyl]-2,4-dimethyl-1H-pyrrol-3-yl]carbonyl]-2-(1-pyrrolidinylmethyl)pyrrolidine (PHA 665752), sodium succinate dibasic, 4,4prime-Bis(4-aminophenoxy)biphenyl, etc.

In some cases, the Src inhibitor comprises N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib), N-(5-chloro-1,3-benzodioxol-4-yl)-7-(2-(4-methylpiperazin-1-yl)ethoxy)-5-(tetrahydro-2H-pyran-4-yloxy)quinazolin-4-amine (saracatinib), 4-[(2,4-dichloro-5-methoxyphenyl)amino]-6-methoxy-7-[3-(4-methylpiperazin-1-yl)propoxy]quinoline-3-carbonitrile (bosutinib), (4-Amino-5-(4-methylphenyl)-7-(t-butyl)pyrazolo[3,4-d]-pyrimidine), PP2 (4-chlorophenyl)-7-(t-butyl)pyrazolo[3,4-d]-pyrimidine) (PP1), 1-tert-butyl-3-(4-chlorophenyl)pyrazolo[3,4-d]pyrimidin-4-amine (PP2), 6-(2,6-dichlorophenyl)-2-{[3-(hydroxymethyl)phenyl]amino}-8-methyl-7H,8H-pyrido[2,3-d]pyrimidin-7-one (PD1663266), (E)-N-[4-[3-chloro-4-(pyridin-2-ylmethoxy)anilino]-3-cyano-7-ethoxyquinolin-6-yl]-4-(dimethylamino)but-2-enamide (neratinib), 3-(2-imidazo[1,2-b]pyridazin-3-ylethynyl)-4-methyl-N-[4-[(4-methylpiperazin-1-yl)methyl]-3-(trifluoromethyl)phenyl]benzamide (ponatinib), (E)-N-[4-(3-chloro-4-fluoroanilino)-3-cyano-7-ethoxyquinolin-6-yl]-4-(dimethylamino)but-2-enamide (pelitinib), N-benzyl-2-[5-[4-(2-morpholin-4-ylethoxy)phenyl]pyridin-2-yl]acetamide (Tirbanibulin), 4-methyl-3-[(2-methyl-6-pyridin-3-ylpyrazolo[3,4-d]pyrimidin-4-yl)amino]-N-[3-(trifluoromethyl)phenyl]benzamide (NVP-BHG712), (2S,3S)-2,3-dihydroxybutanedioic acid; 6-(4-methylpiperazin-1-yl)-N-(5-methyl-1H-pyrazol-3-yl)-2-[(E)-2-phenylethenyl]pyrimidin-4-amine (ENMD-2076), 4-[4-[(5-tert-butyl-2-quinolin-6-ylpyrazol-3-yl)carbamoylamino]-3-fluorophenoxy]-N-methylpyridine-2-carboxamide (Rebastinib) or any combination thereof.

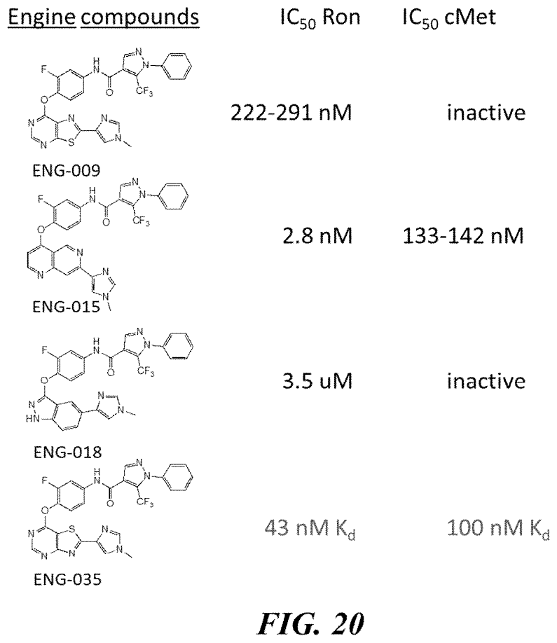

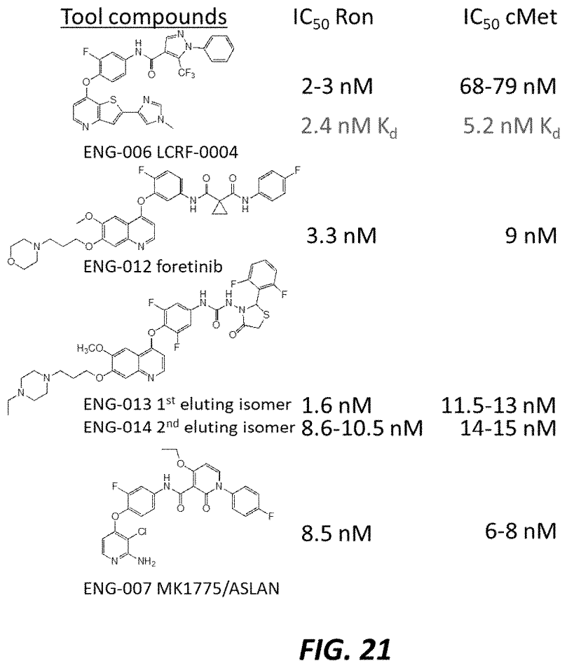

In some cases, the Ron inhibitor comprises N-[4-[(2-amino-3-chloro-4-pyridinyl)oxy]-3-fluorophenyl]-4-ethoxy-1-(4-fluorophenyl)-2-oxo-3-pyridinecarboxamide (BMS777607), N1′-[3-fluoro-4-[[6-methoxy-7-(3-morpholinopropoxy)-4-quinolyl]oxy]phenyl]-N1-(4-fluorophenyl)cyclopropane-1,1-dicarboxamide (Foretinib), N-(3-fluoro-4-((2-(1-methyl-1H-imidazol-4-yl)thiazolo[5,4-d]pyrimidin-7-yl)oxy)phenyl)-1-phenyl-5-(trifluoromethyl)-1H-pyrazole-4-carboxamide (ENG-009, FIG. 20), N-(3-fluoro-4-((7-(1-methyl-1H-imidazol-4-yl)-1,6-naphthyridin-4-yl)oxy)phenyl)-1-phenyl-5-(trifluoromethyl)-1H-pyrazole-4-carboxamide (ENG-015, FIG. 20), N-(3-fluoro-4-((6-(1-methyl-1H-imidazol-4-yl)-1H-indazol-3-yl)oxy)phenyl)-1-phenyl-5-(trifluoromethyl)-1H-pyrazole-4-carboxamide (ENG-018, FIG. 20), N-(3-fluoro-4-((2-(1-methyl-1H-imidazol-4-yl)thiazolo[4,5-b]pyridin-7-yl)oxy)phenyl)-1-phenyl-5-(trifluoromethyl)-1H-pyrazole-4-carboxamide (ENG-035, FIG. 20), N-(3-fluoro-4-((2-(1-methyl-1H-imidazol-4-yl)thieno[3,2-b]pyridin-7-yl)oxy)phenyl)-1-phenyl-5-(trifluoromethyl)-1H-pyrazole-4-carboxamide (ENG-006, FIG. 21), 1-(2-(2,6-difluorophenyl)-4-oxothiazolidin-3-yl)-3-(4-((7-(3-(4-ethylpiperazin-1-yl)propoxy)-6-methoxyquinolin-4-yl)oxy)-3,5-difluorophenyl)urea (ENG-013, FIG. 21), N-[4-[(2-amino-3-chloro-4-pyridinyl)oxy]-3-fluorophenyl]-4-ethoxy-1-(4-fluorophenyl)-2-oxo-3-pyridinecarboxamide (ENG-007, FIG. 21), or any combination thereof. In some cases, the Ron inhibitor comprises N-(3-fluoro-4-((2-(1-methyl-1H-imidazol-4-yl)thiazolo[5,4-d]pyrimidin-7-yl)oxy)phenyl)-1-phenyl-5-(trifluoromethyl)-1H-pyrazole-4-carboxamide (ENG-009, FIG. 20) or N-(3-fluoro-4-((7-(1-methyl-1H-imidazol-4-yl)-1,6-naphthyridin-4-yl)oxy)phenyl)-1-phenyl-5-(trifluoromethyl)-1H-pyrazole-4-carboxamide (ENG-015, FIG. 20). In some cases, a Ron inhibitor comprises a Ron IC₅₀ of at most 1 micromolar (μM). In some cases, said Ron IC₅₀ reflects the short-form Ron (sfRon) IC₅₀.

In some cases, the small molecule inhibitor may comprise a combination of small molecule inhibitors or derivatives thereof. For example, a small molecule inhibitor may be engineered or modified for dual specificity and may decrease expression or activity of both members of the gene pair. Alternatively or in addition to, a combination of small molecule inhibitors (e.g., a small molecule “cocktail”) may be used to decrease expression or activity of both members of the gene pair. In some cases, a small molecule inhibitor may be administered with another agent type (e.g., protein, RNA molecule, DNA molecule, etc.). In some cases a Ron inhibitor may comprise a cMet IC₅₀ that is at least 50 times greater than a Ron IC₅₀ of said Ron inhibitor.

The small molecule inhibitor may be administered in any useful concentration. For example, a small molecule may be administered at a concentration of about 0.5 nanomolar (nM), about 1 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 micromolar (μM), about 2 μM, about 3 μM, about 4 μM, about 5 μM, about 6 μM, about 7 μM, about 8 μM, about 9 μM, about 10 μM. A small molecule may be administered at a concentration of at least about 0.5 nanomolar (nM), at least about 1 nM, at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 200 nM, at least about 300 nM, at least about 400 nM, at least about 500 nM, at least about 600 nM, at least about 700 nM, at least about 800 nM, at least about 900 nM, at least about 1 micromolar (μM), at least about 2 μM, at least about 3 μM, at least about 4 μM, at least about 5 μM, at least about 6 μM, at least about 7 μM, at least about 8 μM, at least about 9 μM, at least about 10 μM. A small molecule may be administered at a concentration of at most about 10 μM, at most about 9 μM, at most about 8 μM, at most about 7 μM, at most about 6 μM, at most about 5 μM, at most about 4 μM, at most about 3 μM, at most about 2 μM, at most about 1 μM, at most about 900 nM, at most about 800 nM, at most about 700 nM, at most about 600 nM, at most about 500 nM, at most about 400 nM, at most about 300 nM, at most about 200 nM, at most about 100 nM, at most about 90 nM, at most about 80 nM, at most about 70 nM, at most about 60 nM, at most about 50 nM, at most about 40 nM, at most about 30 nM, at most about 20 nM, at most about 10 nM, at most about 1 nM, at most about 0.5 nM, etc. A range of concentrations may be used, e.g., between 22 nM-1 μM). Where more than one small molecule is used, the concentrations may be the same of different for each small molecule used.

The small molecule inhibitor may be administered in any useful dose. For example, a small molecule may be administered at a dose of about 50 micrograms (μg), a dose of about 100 μg, a dose of about 200 μg, a dose of about 300 μg, a dose of about 400 μg, a dose of about 500 μg, a dose of about 750 μg, a dose of about 1 milligram (mg), a dose of about 1.2 mg, a dose of about 1.5 mg, a dose of about 2 mg, a dose of about 3 mg, a dose of about 4 mg, a dose of about 5 mg, a dose of about 6 mg, a dose of about 8 mg, a dose of about 10 mg, a dose of about 12 mg, a dose of about 15 mg, a dose of about 20 mg, a dose of about 25 mg, a dose of about 30 mg, a dose of about 40 mg, a dose of about 50 mg, a dose of about 60 mg, a dose of about 80 mg, a dose of about 100 mg, a dose of about 120 mg, a dose of about 140 mg, a dose of about 160 mg, a dose of about 180 mg, a dose of about 200 mg, a dose of about 225 mg, a dose of about mg, a dose of about 250 mg, a dose of about 275 mg, a dose of about 300 mg, a dose of about 350 mg, a dose of about 400 mg, a dose of about 500 mg, a dose of about 600 mg, a dose of about 800 mg. The small molecule inhibitor may be administered in any useful dose. For example, a small molecule may be administered at a dose of at least 50 micrograms (μg), a dose of at least 100 μg, a dose of at least 200 μg, a dose of at least 300 μg, a dose of at least 400 μg, a dose of at least 500 μg, a dose of at least 750 μg, a dose of at least 1 milligram (mg), a dose of at least 1.2 mg, a dose of at least 1.5 mg, a dose of at least 2 mg, a dose of at least 3 mg, a dose of at least 4 mg, a dose of at least 5 mg, a dose of at least 6 mg, a dose of at least 8 mg, a dose of at least 10 mg, a dose of at least 12 mg, a dose of at least 15 mg, a dose of at least 20 mg, a dose of at least 25 mg, a dose of at least 30 mg, a dose of at least 40 mg, a dose of at least 50 mg, a dose of at least 60 mg, a dose of at least 80 mg, a dose of at least 100 mg, a dose of at least 120 mg, a dose of at least 140 mg, a dose of at least 160 mg, a dose of at least 180 mg, a dose of at least 200 mg, a dose of at least 225 mg, a dose of at least mg, a dose of at least 250 mg, a dose of at least 275 mg, a dose of at least 300 mg, a dose of at least 350 mg, a dose of at least 400 mg, a dose of at least 500 mg, a dose of at least 600 mg, a dose of at least 800 mg. The small molecule inhibitor may be administered at a dose of at most 800 mg, at a dose of at most 600 mg, at a dose of at most 500 mg, at a dose of at most 400 mg, at a dose of at most 300 mg, at a dose of at most 250 mg, at a dose of at most 225 mg, at a dose of at most 200 mg, at a dose of at most 180 mg, at a dose of at most 160 mg, at a dose of at most 140 mg, at a dose of at most 120 mg, at a dose of at most 100 mg, at a dose of at most 80 mg, at a dose of at most 60 mg, at a dose of at most 50 mg, at a dose of at most 40 mg, at a dose of at most 30 mg, at a dose of at most 25 mg, at a dose of at most 20 mg, at a dose of at most 15 mg, at a dose of at most 12 mg, at a dose of at most 10 mg, at a dose of at most 8 mg, at a dose of at most 6 mg, at a dose of at most 5 mg, at a dose of at most 4 mg, at a dose of at most 3 mg, at a dose of at most 2 mg, at a dose of at most 1.5 mg, at a dose of at most 1.2 mg, a dose of at most 1 mg, a dose of at most 750 μg, a dose of at most 600 μg, a dose of at most 500 μg, a dose of at most 400 μg, a dose of at most 350 μg, a dose of at most 300 μg, a dose of at most 250 μg, a dose of at most 200 μg, a dose of at most 180 μg, a dose of at most 150 μg, a dose of at most 120 μg, a dose of at most 100 μg, a dose of at most 80 μg, a dose of at most 50 μg, a dose of at most 20 μg, a dose of at most 10 μg. Where more than one small molecule is used, the dosages may be the same of different for each small molecule used. Where more than one small molecule is used, the dosing frequencies may be the same or different for each molecule used.

In some cases, the one or more agents used to cause a decrease in expression or activity of one or both members of a gene pair may comprise a nucleic acid molecule, e.g., a RNA molecule. The RNA molecule can comprise any suitable RNA molecule and size sufficient to decrease the expression level or activity of one or both members of a gene pair. The RNA molecule may comprise a small hairpin RNA (shRNA) molecule, a small interfering RNA (siRNA), a microRNA (miRNA), or other useful RNA molecule. In some examples, the RNA molecule may comprise a messenger RNA (mRNA), transfer RNA (tRNA), ribosomal RNAs (rRNA), small nuclear RNA (snRNA), piwi-interacting (piRNA), non-coding RNA (ncRNA), long non-coding RNA, (lncRNA), and fragments of any of the foregoing. The RNA molecule may be single-stranded, double-stranded, or partially single- or double-stranded.

It will be appreciated that one or more agents (e.g., peptides, RNA molecules, protein-nucleic acid complexes) are listed as examples and that a combination of agent types may be used to treat the subject. For instance, administering one or more different types of agents may be used to decrease the expression or activity of both members of one or more gene pairs. For example, a protein or peptide may be co-administered with a small molecule, an RNA molecule, a DNA molecule, or a complexed molecule (e.g., protein-nucleic acid molecule). Similarly, a RNA molecule may be administered with a small molecule, a DNA molecule, or a complexed molecule. In another example, a small molecule may be co-administered with a DNA molecule or a complexed molecule. These combinations are non-limiting examples of different combinations of agents that may be used to treat the subject having or suspected of having cancer.

In some cases, the cancer selected to be treated may comprise breast cancer. In some cases, the breast cancer may be triple-negative breast cancer. In other cases, the cancer selected to be treated may comprise an aggressive cancer type for which few biomarkers or target genes to which the cancer cells are sensitive are known. In some cases, the cancer comprises increased Src expression levels. In some cases, the cancer comprises increased Ron expression levels. In some cases, the cancer comprises increased Src and Ron expression levels. In some cases, the cancer comprises a constitutively active Src. In some cases, the cancer comprise a short-form Ron (sfRon).

Table 1 provides a list of gene pairs for which a decrease in expression or activity of both members may lead to cell death. GeneA and GeneB refer to individual members of the gene pair GeneA_GeneB. The Modified GI is a modified gene interaction score for the gene pairs. The gene pairs may be synthetic lethal gene pairs, such that a decreased expression or activity of only one member may not lead to cell death but decreased expression or activity of both members may cause or lead to cell death.

TABLE 1
List of gene pairs and modified gene interaction
scores for each gene pair.

| | GeneA_GeneB | Modified GI |
|---|---|---|
| 1 | SRC_YES1 | −3.165 |
| 2 | RON_SRC | −2.405 |
| 3 | AXL_PTK2 | −2.396 |
| 4 | ERBB2_WEE1 | −2.353 |
| 5 | ERBB3_ABL2 | −2.173 |
| 6 | PTK2_TYRO3 | −2.121 |
| 7 | RON_AXL | −2.098 |
| 8 | EPHB3_ABL2 | −2.073 |
| 9 | PTK2B_PTK2 | −2.005 |
| 10 | JAK2_WEE1 | −1.993 |
| 11 | EPHA5_SRC | −1.889 |
| 12 | SRC_EPHB6 | −1.859 |
| 13 | WEE1_TNK1 | −1.844 |
| 14 | WEE1_ERBB3 | −1.837 |
| 15 | EPHA5_PTK2 | −1.671 |

In another aspect, disclosed herein is a composition for treating a subject having or suspected of having a cancer, comprising a formulation comprising at least one agent present in an amount that is effective to cause a decrease in expression or activity of one or more gene pairs selected from Table 1.

In some cases, the composition comprises a DNA construct which may comprise a guide RNA (gRNA) sequence that may be used to direct a protein (e.g., Cas protein) to one or both of the members of at least one gene pair. The DNA construct may comprise at least one gRNA sequence, each of which may direct the protein (e.g., Cas protein) to a different gene. The DNA construct may comprise a RNA sequence, a DNA sequence, or a combination thereof. In some cases, the DNA construct comprises: (i) a first gRNA sequence, which may be used to direct an endonuclease (e.g., Cas protein) to a targeted location or gene locus for one member of the gene pair, (ii) a second gRNA sequence, which may be used to direct an endonuclease (e.g., Cas protein) to another targeted location or gene locus for the other member of the gene pair, (iii) a first sequence (e.g., a DNA sequence) corresponding to one member of the gene pair (e.g., a gene replacement), and (iv) a second sequence (e.g., DNA sequence) corresponding to the other member of the gene pair (e.g., a gene replacement). It will be appreciated, that different combinations of RNA sequences and DNA sequences may be used in the DNA construct. Moreover, other functional sequences may be included in the DNA sequence, including, but not limited to, a barcode sequence, a tag, or other identifying sequence, a primer sequence, a restriction site, a transposition site, etc.

The endonuclease complex may comprise an endonuclease, e.g., a Cas protein, or other nucleic acid-interacting enzyme (e.g., ligase, helicase, reverse transcriptase, transcriptase, polymerase, etc.). The Cas protein may comprise any Cas type (e.g., Cas I, Cas IA, Cas IB, Cas IC, Cas ID, Cas IE, Cas IF, Cas IU, Cas III, Cas IIIA, Cas IIIB, Cas IIIC, Cas IIID, Cas IV, Cas IVA, Cas IVB, Cas II, Cas IIA, Cas IIB, Cas IIC, Cas V, Cas VI). In some instances, the Cas protein may comprise other proteins (e.g., a fusion protein) and may comprise an additional enzyme that may associate with a nucleic acid molecule (e.g., ligase, transcriptase, transposase, nuclease, endonuclease, reverse transcriptase, polymerase, helicase, etc.). The endonuclease complex may be delivered exogenously or may be encoded in the DNA construct for transcription and translation within the cell.

The DNA construct may comprise a RNA sequence, a DNA sequence, or a combination thereof In some cases, the DNA construct comprises: (i) a first gRNA sequence, which may be used to direct an endonuclease (e.g., Cas protein) to a targeted location or gene locus for one member of the gene pair, (ii) a second gRNA sequence, which may be used to direct an endonuclease (e.g., Cas protein) to another targeted location or gene locus for the other member of the gene pair, (iii) a first sequence (e.g., a DNA sequence) corresponding to one member of the gene pair (e.g., a gene replacement), and (iv) a second sequence (e.g., DNA sequence) corresponding to the other member of the gene pair (e.g., a gene replacement). It will be appreciated, that different combinations of RNA sequences and DNA sequences may be used in the DNA construct. Moreover, other functional sequences may be included in the DNA sequence, including, but not limited to, a barcode sequence, a tag, or other identifying sequence, a primer sequence, a restriction site, a transposition site, etc.

In some cases, the composition comprises one or more agents used to cause a decrease in expression or activity of one or both members of a gene pair and may comprise a protein or peptide. For example, the one or more agents may comprise an antibody, an antibody fragment, a hormone, a ligand, or an immunoglobulin. The protein or peptide may be naturally occurring or may be synthetic. The protein may be an engineered variant of a protein (e.g., recombinant protein), or fragment thereof. The protein may be subjected to other modifications, e.g., post-translational modifications, including but not limited to: glycosylation, acylation, prenylation, lipoylation, alkylation, amidation, acetylation, methylation, formylation, butyrylation, carboxylation, phosphorylation, malonylation, hydroxylation, iodination, propionylation, S-nitrosylation, S-glutationylation, succinylation, sulfation, glycation, carbamylation, carbonylation, biotinylation, carbamylation, oxidation, pegylation, sumoylation, ubiquitination, ubiquitylation, racemization, etc. One or more modifcations may be made to the protein or peptide.

In some cases, the composition may comprise one or more agents used to cause a decrease in expression or activity of one or both members of a gene pair and may comprise a small molecule. The small molecule may be configured to decrease the expression level or activity level of one or both members of a gene pair. In some cases, the small molecule may directly interact with one or both members of the gene pair. For example, the small molecule may inhibit the protein or proteins encoded by one or both members of the gene pair, respectively. Alternatively or in addition to, the small molecule may inhibit an upstream effector or downstream protein in a signaling pathway in which one or both members of the gene pair interact.

In some cases, the small molecule inhibitor may comprise a Src inhibitor, e.g., Dasatinib, Quercetin, PP1 or PP2 kinase inhibitor, Saracatinib, Bosutinib, or KX2-391. In some cases, the small molecule inhibitor may comprise a Yes inhibitor, e.g., SU-6656, PD173955, PRT062607, or Saracatinib. In some cases, the small molecule inhibitor may comprise a Ron inhibitor, e.g., BMS777607, Foretinib, PHA 665752, sodium succinate dibasic, 4,4prime-Bis(4-aminophenoxy)biphenyl, etc. In some cases, the small molecule inhibitor may comprise a combination of small molecule inhibitors or derivatives thereof. For example, a small molecule inhibitor may be engineered or modified for dual specificity and may decrease expression of both members of the gene pair. Alternatively or in addition to, a combination of small molecule inhibitors may be used to decrease expression or activity of both members of the gene pair.

A small molecule inhibitor may inhibit a secondary target. The secondary target may comprise a protein, macromolecular complex, a ribozyme, or any combination thereof. The secondary target may be related to the primary target (e.g., Src for an Src inhibitor). For example, a Ron or Src inhibitor may comprise a protein kinase as a secondary target. The small molecule inhibitor may have a similar or lower affinity (e.g., K_{d}) or inhibitory activity (e.g., IC₅₀) toward the secondary target. Such secondary target affinity or inhibitory activity may enhance the efficacy of the small molecule inhibitor for treating a cancer, such as triple negative breast cancer. A Ron or Src small molecule inhibitor may comprise a protein kinase secondary target, such as Tyro3, cKit, EGFR, JAK2, or PDK1.

Conversely, a small molecule inhibitor may predominantly target a single protein. In particular instances, a small molecule inhibitor may predominantly target a single protein kinase, such as Ron, Src, Yes1. For example, the IC₅₀ of a small molecule inhibitor for its primary target may be at least 10 times, at least 20 times, at least 25 times, at least 40 times, at least 50 times, at least 100 times, or at least 200 times lower than the IC₅₀ of the small molecule inhibitor for a secondary target. As a further example, a small molecule inhibitor may comprise an IC₅₀ of less than 1 μM, 5 μM, 10 μM, less than 20 μM, less than 25 μM, less than 40 μM, less than 50 μM, or less than 100 μM for only one protein from a class of proteins (e.g., a small molecule inhibitor may comprise an IC₅₀ below 1 μM for only a single protein kinase).

In some cases, the composition may comprise one or more agents used to cause a decrease in expression or activity of one or both members of a gene pair and may comprise a nucleic acid molecule, e.g., a RNA molecule. The RNA molecule comprise any suitable RNA molecule and size sufficient to decrease the expression level or activity of one or both members of a gene pair. The RNA molecule may comprise a small hairpin RNA (shRNA) molecule, a small interfering RNA (siRNA), a microRNA (miRNA), or other useful RNA molecule. In some examples, the RNA molecule may comprise a messenger RNA (mRNA), transfer RNA (tRNA), ribosomal RNAs (rRNA), small nuclear RNA (snRNA), piwi-interacting (piRNA), non-coding RNA (ncRNA), long non-coding RNA, (lncRNA), and fragments of any of the foregoing. The RNA molecule may be single-stranded, double-stranded, or partially single- or double-stranded.

It will be appreciated that one or more agents (e.g., peptides, RNA molecules, protein-nucleic acid complexes) are listed as examples and that a combination of agent types may be comprised in the composition. For instance, the composition may comprise one or more different types of agents that may be used to decrease the expression or activity of both members of one or more gene pairs. For example, the composition may comprise a protein or peptide that may be co-administered with a small molecule, an RNA molecule, a DNA molecule, or a complexed molecule (e.g., protein-nucleic acid molecule). Similarly, the composition may comprise a RNA molecule that may be administered with a small molecule, a DNA molecule, or a complexed molecule. In another example, a small molecule may be co-administered with a DNA molecule or a complexed molecule. These combinations are non-limiting examples of different combinations of agents that may be used in the compositions described herein.

### Ron-Selective Inhibitors

In one aspect, provided herein is a compound of Formula (I), or a pharmaceutically acceptable salt, solvate, tautomer, or N-oxide thereof:

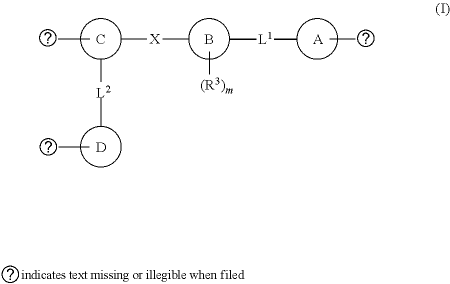

wherein,
ring A is heterocycloalkyl, aryl or monocyclic heteroaryl;
L¹ is a bond, —C(═O)—, —C(═O)—NR²—, —C(═O)—O—, —C(═O)—S—, —NR²—C(═O)—, —O—C(═O)—, —S—C(═O)—, —NR²—C(═O)—NR²—, —NR²—C(═S)—NR²—, —NR²—C(═O)—O—, —O—C(═O)—O—, —NR²—, —O—, —S—, or —S(═O)₂—;
ring B is aryl or heteroaryl;
X is a bond, —NR²—, —O—, —S—, —S(═O)₂—, —C(═O)—, —C(═O)—NR²—, —C(═O)—O—, —C(═O)—S—, —NR²—C(═O)—, —O—C(═O)—, or —S—C(═O)—;
ring C is 5-membered heteroaromatic, bicyclic fused aromatic, or bicyclic fused heteroaromatic;
L² is a bond, —O—, —NR²—, —S—, or —S(═O)₂—, or alternatively L² is absent and ring C is fused with ring D;
ring D is aryl, heteroaryl, C₃-C₆ cycloalkyl, or C₂-C₅ heterocycloalkyl;
each instance of R¹ is independently selected from the group consisting of optionally substituted aryl, optionally substituted heteroaryl, halogen, —NO₂, —NR⁶R⁷, hydroxyl, —C(═O)—R⁸, C₁-C₄ alkyl, C₁-C₄ alkoxy, C₁-C₄ haloalkyl, C₁-C₄ heteroalkylene, and C₁-C₄ haloalkoxy;
each instance of R² is independently selected from the group consisting of hydrogen, C₁-C₄ alkyl, C₁-C₄ hydroxyalkyl, C₁-C₄ heteroalkylene. and C₁-C₄ haloalkyl;
each instance of R³ is independently selected from the group consisting of halogen, —NO₂, —NR⁶R⁷, hydroxyl, —C(═O)—R⁸, C₁-C₄ alkyl, C₁-C₄ alkoxy, C₁-C₄ heteroalkylene, C₁-C₄ haloalkyl, and C₁-C₄ haloalkoxy;
each instance of R⁴ is independently selected from the group consisting of optionally substituted aryl, optionally substituted heteroaryl, optionally substituted C₃-C₆ cycloalkyl, optionally substituted C₂-C₅ heterocycloalkyl, halogen, —NR⁶R⁷, hydroxyl, —C(═O)—R⁸, C₁-C₄ alkyl, C₁-C₄ alkoxy, C₁-C₄ haloalkyl, C₁-C₄ heteroalkylene, and C₁-C₄ haloalkoxy, or two R⁴ are taken together to form a cycloalkyl, a heterocycloalkyl, an aryl, or a heteroaryl;
each instance of R⁵ is independently selected from the group consisting of halogen, —NR⁶R⁷, hydroxyl, —C(═O)—R⁸, C₁-C₄ alkyl, C₁-C₄ hydroxyalkyl, C₁-C₄ heteroalkylene, C₁-C₄ alkoxy, C₁-C₄ haloalkyl, and C₁-C₄ haloalkoxy;
each instance of R⁶ and R⁷ is independently selected from the group consisting of hydrogen, C₁-C₄ alkyl, C₁-C₄ haloalkyl, and C₁-C₄ hydroxyalkyl, or R⁶ and R⁷ are taken together with the nitrogen atom to which they are attached to form an optionally substituted heterocycloalkyl;
each instance of R⁸ is independently selected from the group consisting of hydrogen, hydroxyl, NR⁶R⁷, C₁-C₄ alkyl, C₁-C₄ alkoxy, C₁-C₄ haloalkyl, and C₁-C₄ haloalkoxy;
m is 0, 1, 2;
n is 0, 1, 2, or 3;
and each instance of 1 is independently taken to be 0, 1, or 2.

In some cases, ring A is aryl or heteroaryl. In some cases, ring A is heteroaryl. In some cases, ring A is 5-membered heteroaryl. In some cases, ring A is pyrrole, imidazole, pyrazole, triazole, thiazole, isoxazole, thiazolidone, or oxadiazole. In some cases, ring A is imidazole, thiazolidone, or pyrazole. In some cases, ring A is thiazolidone or pyrazole. In some cases, ring A is pyrazole. In some cases, ring A is thiazolidone. In some cases, ring A is

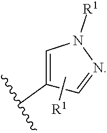

In some cases, ring A is

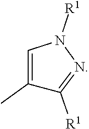

In some cases, ring A is

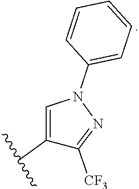 [SMILES: *c1cn(-c2ccccc2)nc1C(F)(F)F]

In some cases, ring A is

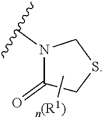

In some cases, L¹ is —NR²—C(═O)—, —O—C(═O)—, or —S—C(═O)—. In some cases, L¹ is —NR²—C(═O)—. In some cases, L¹ is —NH—C(═O)—.

In some cases, ring B is monocyclic aryl or monocyclic heteroaryl. In some cases, ring B is phenyl, pyridine, pyrimidine, pyrrole, pyrazole, imidazole, triazole, thiazole, oxazole, thiophene, or furan. In some cases, ring B is phenyl. In some cases, ring B is

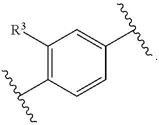

In some cases, ring B is

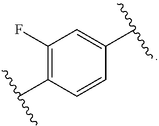 [SMILES: *c1ccc(*)c(F)c1]

In some cases, X is —NH—, —N(CH₃)—, —CH₂—, —O—, —S—, or —S(═O)₂—. In some cases, X is —O— or —S—. In some cases, X is —O—.

In some cases, ring C is benzothiazole, thiazolo[5,4-c]pyridine, thiazolo[4,5-b]pyridine, thiazolo[4,5-d]pyrimidine, thiazolo[5,4-d]pyrimidine, thiazolo[5,4-b]pyridine, thiazolo[4,5-c]pyridine, naphthalene, pyrrolizine, isoindole, indolizine, quinoline, an isoquinonline, 4H-quinolizine, indazole, quinoxaline, quinazoline, phthalazine, cinnoline, naphthyridine, 1H-indazole, purine, pteridine, pyrrole, pyrazole, imidazole, triazole, thiazole, oxazole, thiophene, or furan. In some cases, ring C is thiazolo[5,4-c]pyridine, thiazolo[4,5-b]pyridine, thiazolo[4,5-d]pyrimidine, thiazolo[5,4-d]pyrimidine, thiazolo[5,4-b]pyridine, thiazolo[4,5-c]pyridine, naphthalene, quinoline, quinazoline, quinoxaline, 1,5-naphthyridine, 2,6-naphthyridine, 1,6-naphthyridine, 1H-indazole, pyrazole, pyrrole, or imidazole. In some cases, ring C is benzothiazole, thiazolo[5,4-c]pyridine, thiazolo[4,5-b]pyridine, thiazolo[4,5-d]pyrimidine, 1,5-naphthyridine, 2,6-naphthyridine, 1,6-naphthyridine, pyrrole, pyrazole, imidazole, or thiazole. In some cases, ring C is thiazolo[5,4-d]pyrimidine, thiazolo[4,5-d]pyrimidine, 1,6-naphthyridine, quinoline, 2,6-naphthyridine, pyrazole, or imidazole. In some cases, ring C is optionally R⁴ substituted

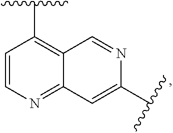 [SMILES: *c1cc2nccc(*)c2cn1]

optionally R⁴ substituted

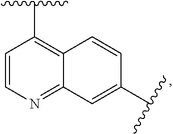 [SMILES: *c1ccc2c(*)ccnc2c1]

optionally R⁴ substituted

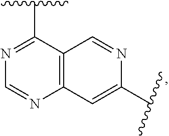 [SMILES: *c1cc2ncnc(*)c2cn1]

optionally R⁴ substituted

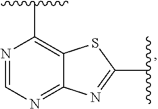 [SMILES: *c1nc2ncnc(*)c2s1]

optionally R⁴ substituted

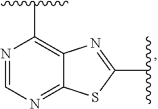 [SMILES: *c1nc2c(*)ncnc2s1]

optionally R⁴ substituted

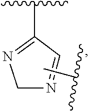

optionally R⁴ substituted

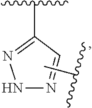

or optionally R⁴ substituted

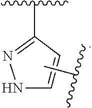

In some cases, ring C is optionally R⁴ substituted

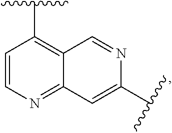 [SMILES: *c1cc2nccc(*)c2cn1]

optionally R⁴ substituted

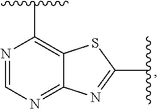 [SMILES: *c1nc2ncnc(*)c2s1]

optionally R⁴ substituted

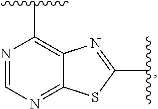 [SMILES: *c1nc2c(*)ncnc2s1]

or optionally R⁴ substituted

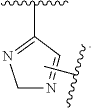

In some cases, L² is absent and ring C and ring D are taken together to form a fused bicyclic or tricyclic structure. In some cases, the bicyclic structure is selected from the group consisting of

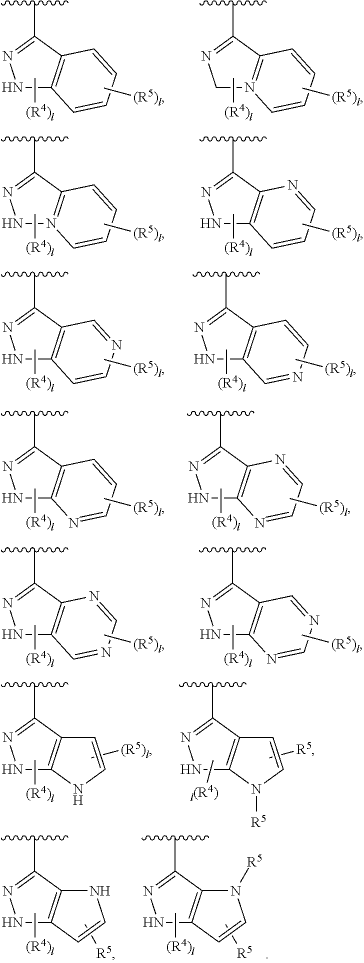

In some cases, the bicyclic structure is selected from the group consisting of

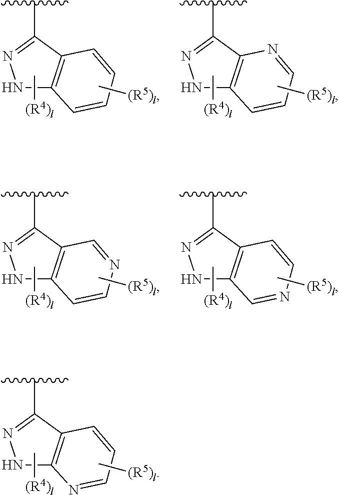

In some cases, the bicyclic structure is

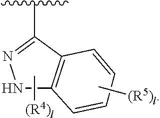

In some cases, L² is a bond, —O—, —NR²—, or —CH₂—. In some cases, L² is a bond.

In some cases, ring D is aryl or a 5- or 6-membered heteroaryl. In some cases ring D is 5- or 6-membered heteroaryl. In some cases, ring D is pyrrole, pyrazole, imidazole, triazole, thiazole, thiophene, oxazole, or furan. In some cases, ring D is pyrrole, pyrazole, imidazole, or triazole. In some cases, ring D is pyrazole or imidazole. In some cases, ring D is

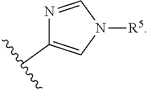

In some cases, ring D is

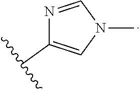 [SMILES: *c1cn(C)cn1]

In some cases, each instance of R¹ is independently selected from the group consisting of optionally substituted aryl, optionally substituted heteroaryl, halogen, C₁-C₄ alkyl, and C₁-C₄ haloalkyl. In some cases, each instance of le is selected from the group consisting of C₁-C₄ haloalkyl, halogen, C₁-C₄ alkyl, and optionally substituted aryl. In some cases, each instance of R¹ is selected from the group consisting of C₁-C₄ haloalkyl and optionally substituted aryl. In some cases, each instance of R¹ is selected from the group consisting of C₁ haloalkyl and phenyl.

In some cases, each instance of R² is independently selected from the group consisting of hydrogen and C₁-C₄ alkyl. In some cases, each instance of R² is hydrogen.

In some cases, each instance of R³ is independently selected from the group consisting of halogen, —NR⁶R⁷, hydroxyl, —C(═O)—R⁸, C₁-C₄ alkyl, and C₁-C₄ haloalkyl. In some cases, each instance of R³ is independently selected from the group consisting of halogen, hydroxyl, C₁-C₄ alkyl, and C₁-C₄ haloalkyl. In some cases, each instance of R³ is independently selected from the group consisting of halogen and hydroxyl. In some cases, each instance of R³ is halogen. In some cases, each instance of R³ is F.

In some cases, each instance of R⁴ is independently selected from the group consisting of optionally substituted aryl, optionally substituted heteroaryl, optionally substituted C₃-C₆ cycloalkyl, optionally substituted C₂-C₅ heterocycloalkyl, halogen, and C₁-C₄ haloalkyl. In some cases, each instance of R⁴ is independently selected from the group consisting of optionally substituted aryl, optionally substituted heteroaryl, and optionally substituted C₂-C₅ heterocycloalkyl. In some cases, each instance of R⁴ is independently selected from the group consisting of optionally substituted aryl and optionally substituted heteroaryl. In some cases, each instance of R⁴ is selected from the group consisting of optionally substituted phenyl, optionally substituted pyridine, optionally substituted pyrimidine, optionally substituted pyrrole, optionally substituted pyrazole, optionally substituted imidazole, optionally substituted triazole, optionally substituted thiazole, optionally substituted thiophene, optionally substituted oxazole, and optionally substituted furan. In some cases, each instance of R⁴ is selected from the group consisting of optionally substituted phenyl, optionally substituted pyrrole, optionally substituted pyrazole, and optionally substituted imidazole. In some cases, each instance of R⁴ is selected from the group consisting of optionally substituted pyrazole and optionally substituted imidazole. In some cases, each instance of R⁴ is optionally substituted imidazole. In some cases, each instance of R⁴ is

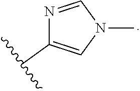 [SMILES: *c1cn(C)cn1]

In some cases, each instance of R⁵ is independently selected from the group consisting of halogen, hydroxyl, C₁-C₄ alkyl and C₁-C₄ haloalkyl. In some cases, each instance of R⁵ is independently selected from the group consisting of halogen and C₁-C₄ alkyl. In some cases, each instance of R⁵ is C₁-C₄ alkyl.

In some cases, each instance of R⁶ and R⁷ is independently selected from the group consisting of hydrogen and C₁-C₄ alkyl. In some cases, each instance of R⁶ is independently selected from the group consisting of hydrogen, C₁-C₄ alkyl, C₁-C₄ haloalkyl, or C₁-C₄ hydroxyalkyl, and each instance of R⁷ is hydrogen. In some cases, each instance of R⁶ is independently selected from the group consisting of C₁-C₄ alkyl, C₁-C₄ haloalkyl, or C₁-C₄ hydroxyalkyl, and each instance of R⁷ is hydrogen. In some cases, each instance of R⁶ and R⁷ is independently selected from the group consisting of hydrogen and C₁-C₄ alkyl. In some cases, each instance of R⁶ and R⁷ is hydrogen.

In some cases, each instance of R⁸ is independently selected from the group consisting of hydroxyl, NR⁶R⁷, C₁-C₄ alkyl, and C₁-C₄ alkoxy. In some cases, each instance of R⁸ is independently selected from the group consisting of hydroxyl and C₁-C₄ alkoxy.

In some cases, m is 1 or 2, n is 2, and 1 are each independently either 0 and 1. In some cases, m is 1, n is 2, and 1 are each independently either 0 and 1. In some cases, m is 1, n is 2, and 1 is 0.

‘Optionally substituted’ may denote substitution with oxo, carboxylate, nitrile, nitro, hydroxyl, thiooxy, alkyl, alkylene, alkoxy, alkoxyalkyl, alkylcarbonyl, alkyloxycarbonyl, aryl, aralkyl, arylcarbonyl, aryloxycarbonyl, aralkylcarbonyl, aralkyloxycarbonyl, aryloxy, cycloalkyl, cycloalkylalkyl, cycloalkylcarbonyl, cycloalkylalkylcarbonyl, cycloalkyloxycarbonyl, heterocyclyl, heteroaryl, dialkylamines, arylamines, alkylarylamines, diarylamines, perfluoroalkyl or perfluoroalkoxy, for example, trifluoromethyl or trifluoromethoxy. “Substituted” can also denote replacement of a hydrogen atom by a higher-order bond (e.g., a double- or triple-bond) to a heteroatom such as oxygen in oxo, carbonyl, carboxyl, and ester groups; and nitrogen in groups such as imines, oximes, hydrazones, and nitriles. In some cases, ‘optionally substituted’ denotes substitution with one or more instances of halogen, —NO₂, sulfuryl, phosphoryl, —NH₂, hydroxyl, —C(═O)—O-Me, —C(═O)—N(Me)₂, C₁-C₄ alkyl, C₁-C₄ alkoxy, C₁-C₄ haloalkyl, and C₁-C₄ haloalkoxy, or any combination thereof. In some cases, ‘optionally substituted’ denotes substitution with one or more instances of halogen, —NO₂, —NH₂, hydroxyl, —C(═O)—O-Me, —C(═O)—N(Me)₂, C₁-C₄ alkyl, C₁-C₄ alkoxy, C₁-C₄ haloalkyl, and C₁-C₄ haloalkoxy, or any combination thereof. In some cases, ‘optionally substituted’ denotes substitution with one or more instances of halogen, —NH₂, hydroxyl, C₁-C₄ alkyl, C₁-C₄ haloalkyl, or any combination thereof. In some cases, ‘optionally substituted’ denotes substitution with one or more instances of halogen, C₁ haloalkyl, methyl, or any combination thereof. In some cases, ‘optionally substituted’ denotes substitution with one or more instances of —F, —CF₃, methyl, or any combination thereof. In some cases, no optional substitutions are present.

In some cases, a compound or a pharmaceutically acceptable salt, solvate, tautomer, or N-oxide thereof of Formula (I) comprises the structure of Formula (Ia)

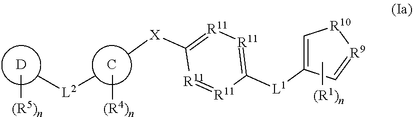

wherein, R⁹ is N, CH, or CR¹, R¹⁰ is NH or NR¹, and each instance of R¹¹ N, CH, or CR³.

In some cases, R⁹ is N, R¹⁰ is NR¹, and at least two R¹¹ are CH. In some cases, R⁹ is N, R¹⁰ is NR¹, and at least three R¹¹ are CH. In some cases, R⁹ is N, R¹⁰ is NR¹, three R¹¹ are CH, and one R¹¹ is CR³.

In some cases, a compound or a pharmaceutically acceptable salt, solvate, tautomer, or N-oxide thereof of Formula (Ia) comprises the structure of Formula (Ib)

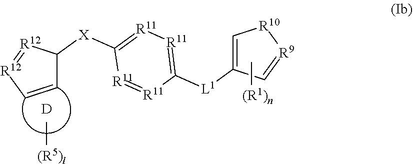

wherein ring D is fused with ring C, and each instance of R¹² is independently selected from N, CH, and CR⁴. In some cases, both instances of R¹² are N. In some cases, one instance of R¹² is N and one instance of R¹² is CH or CR⁴. In some cases, both instances of R¹² are CR⁴. In some cases, both instances of R¹² are CH. In some cases, both instances of R¹² are CR⁴, wherein the two instances of R⁴ are taken together to form an aryl or heteroaryl group.

In some cases, a compound or a pharmaceutically acceptable salt, solvate, tautomer, or N-oxide thereof of Formula (Ia) comprises the structure of Formula (Ic)

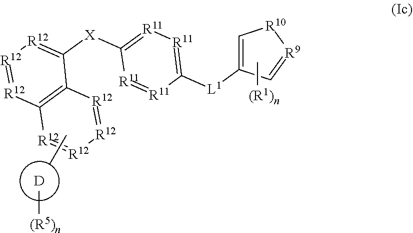

wherein each instance of R¹² is independently selected from N, CH, and CR⁴. In some cases, one or two instances of R¹² are N, and the remainder of R¹² are CH or CR⁴. In some cases, two instances of R¹² are N, and the remainder of R¹² are CH or CR⁴. In some cases, two instances of R¹² are N, and the remainder of R¹² are CH. In some cases, two instances of R¹² are CR⁴, wherein the two instances of R⁴ are taken together to form an aryl or heteroaryl group.

In some cases, a compound or a pharmaceutically acceptable salt, solvate, tautomer, or N-oxide thereof of Formula (Ia) comprises the structure of Formula (Id)

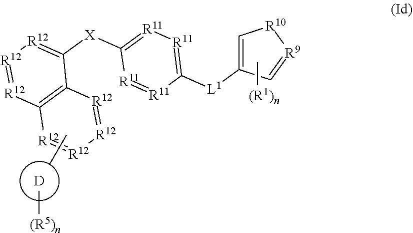

wherein each instance of R¹² is independently selected from the group consisting of N, CH, and CR⁴. In some cases, one or two instances of R¹² are N, and the remainder of R¹² are CH or CR⁴. In some cases, two instances of R¹² are N, and the remainder of R¹² are CH or CR⁴. In some cases, two instances of R¹² are N, and the remainder of R¹² are CH. In some cases, two instances of R¹² are CR⁴, wherein the two instances of R⁴ are taken together to form an aryl or heteroaryl group.

In some cases, a compound or a pharmaceutically acceptable salt, solvate, tautomer, or N-oxide thereof of Formula (Ia) comprises the structure of Formula (Ie):

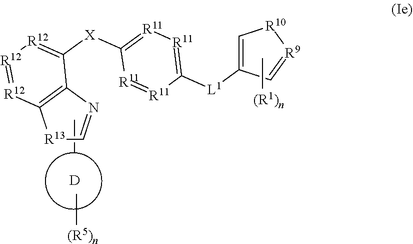

wherein each instance of R¹² is independently selected from the group consisting of N, CH, and CR⁴, and R¹³ is NH, NR⁴, O, or S. In some cases, R¹³ is O or S. In some cases, R¹³ is S. In some cases, one or two instances of R¹² are N, and the remainder of R¹² are CH or CR⁴. In some cases, two instances of R¹² are N, and the remainder of R¹² are CH or CR⁴. In some cases, two instances of R¹² are N, and the remainder of R¹² are CH. In some cases, two instances of R¹² are CR⁴, wherein the two instances of R⁴ are taken together to form an aryl or heteroaryl group.

In some cases, a compound or a pharmaceutically acceptable salt, solvate, tautomer, or N-oxide thereof of Formula (Ia) comprises the structure of Formula (Ie):

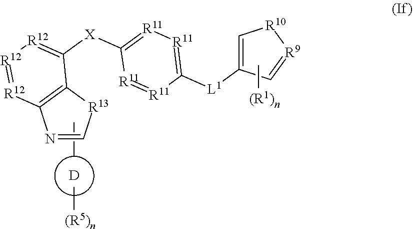

wherein each instance of R¹² is independently selected from the group consisting of N, CH, and CR⁴, and R¹³ is NH, NR⁴, O, or S. In some cases, R¹³ is O or S. In some cases, R¹³ is S. In some cases, one or two instances of R¹² are N, and the remainder of R¹² are CH or CR⁴. In some cases, two instances of R¹² are N, and the remainder of R¹² are CH or CR⁴. In some cases, two instances of R¹² are N, and the remainder of R¹² are CH. In some cases, two instances of R¹² are CR⁴, wherein the two instances of R⁴ are taken together to form an aryl or heteroaryl group.

In some cases, a compound of Formula (I) is N-(3-fluoro-4-((7-(1-methyl-1H-imidazol-4-yl)-1,6-naphthyridin-4-yl)oxy)phenyl)-1-phenyl-5-(trifluoromethyl)-1H-pyrazole-4-carboxamide (ENG-015, FIG. 20), N-(3-fluoro-4-((6-(1-methyl-1H-imidazol-4-yl)-1H-indazol-3-yl)oxy)phenyl)-1-phenyl-5-(trifluoromethyl)-1H-pyrazole-4-carboxamide (ENG-018, FIG. 20), N-(3-fluoro-4-((2-(1-methyl-1H-imidazol-4-yl)thiazolo[4,5-b]pyridin-7-yl)oxy)phenyl)-1-phenyl-5-(trifluoromethyl)-1H-pyrazole-4-carboxamide (ENG-035, FIG. 20), or N-(3-fluoro-4-((2-(1-methyl-1H-imidazol-4-yl)thiazolo[5,4-d]pyrimidin-7-yl)oxy)phenyl)-1-phenyl-5-(trifluoromethyl)-1H-pyrazole-4-carboxamide (ENG-009, FIG. 20).

In some cases, a compound of Formula (I) may comprise Ron inhibitory activity. The compound may comprise a Ron IC₅₀ at most 1 μM. The compound may comprise a Ron IC₅₀ of at most 500 nM. The compound may comprise a Ron IC₅₀ of at most 400 nM. The compound may comprise a Ron IC₅₀ of at most 300 nM. The compound may comprise a Ron IC₅₀ of at most 200 nM. The compound may comprise a Ron IC₅₀ of at most 100 nM. The compound may comprise a Ron IC₅₀ of at most 80 nM. The compound may comprise a Ron IC₅₀ of at most 60 nM. The compound may comprise a Ron IC₅₀ of at most 500 nM. The compound may comprise a Ron IC₅₀ of at most 40 nM. The compound may comprise a Ron IC₅₀ of at most 30 nM. The compound may comprise a Ron IC₅₀ of at most 20 nM. The compound may comprise a Ron IC₅₀ of at most 15 nM. The compound may comprise a Ron IC₅₀ of at most 10 nM. The compound may comprise a Ron IC₅₀ of at most 8 nM. The compound may comprise a Ron IC₅₀ of at most 6 nM. The compound may comprise a Ron IC₅₀ of at most 5 nM. The compound may comprise a Ron IC₅₀ of at most 3 nM. The compound may comprise a Ron IC₅₀ of at most 2 nM. The compound may comprise a Ron IC₅₀ of at most 1 nM.

In some cases, a compound of Formula (I) may comprise cMet inhibitory activity. The compound may comprise a cMet IC₅₀ of at least 50 nM. The compound may comprise a cMet IC₅₀ of at least 100 nM. The compound may comprise a cMet IC₅₀ of at least 200 nM. The compound may comprise a cMet IC₅₀ of at least 400 nM. The compound may comprise a cMet IC₅₀ of at least 500 nM. The compound may comprise a cMet IC₅₀ of at least 600 nM. The compound may comprise a cMet IC₅₀ of at least 800 nM. The compound may comprise a cMet IC₅₀ of at least 1 μM. The compound may comprise a cMet IC₅₀ of at least 2 μM. The compound may comprise a cMet IC₅₀ of at least 3 μM. The compound may comprise a cMet IC₅₀ of at least 4 μM. The compound may comprise a cMet IC₅₀ of at least 5 μM. The compound may comprise a cMet IC₅₀ of at least 8 μM. The compound may comprise a cMet IC₅₀ of at least 10 μM. The compound may comprise a cMet IC₅₀ of at least 12 μM. The compound may comprise a cMet IC₅₀ of at least 15 μM. The compound may comprise a cMet IC₅₀ of at least 20 μM. The compound may comprise a cMet IC₅₀ of at least 25 μM. The compound may comprise a cMet IC₅₀ of at least 40 μM. The compound may comprise a cMet IC₅₀ of at least 50 μM. The compound may comprise a cMet IC₅₀ of at least 80 μM. The compound may comprise a cMet IC₅₀ of at least 100 μM. The compound may comprise a cMet IC₅₀ of at least 120 μM. The compound may comprise a cMet IC₅₀ of at least 150 μM. The compound may comprise a cMet IC₅₀ of at least 200 μM. The compound may comprise a cMet IC₅₀ of at least 250 μM. The compound may comprise a cMet IC₅₀ of at least 300 μM. The compound may comprise a cMet IC₅₀ of at least 400 μM. The compound may comprise a cMet IC₅₀ of at least 500 μM.

In some cases, a compound of Formula (I) may comprise greater Ron inhibitory activity than cMet inhibitory activity. The compound may comprise a cMet IC₅₀ that is at least twice that of a Ron IC₅₀ of said compound. The compound may comprise a cMet IC₅₀ that is at least 5 times greater than a Ron IC₅₀ of said compound. The compound may comprise a cMet IC₅₀ that is at least 10 times greater than a Ron IC₅₀ of said compound. The compound may comprise a cMet IC₅₀ that is at least 20 times greater than a Ron IC₅₀ of said compound. The compound may comprise a cMet IC₅₀ that is at least 25 times greater than a Ron IC₅₀ of said compound. The compound may comprise a cMet IC₅₀ that is at least 50 times greater than a Ron IC₅₀ of said compound. The compound may comprise a cMet IC₅₀ that is at least 100 times greater than a Ron IC₅₀ of said compound. The compound may comprise a cMet IC₅₀ that is at least 200 times greater than a Ron IC₅₀ of said compound. The compound may comprise a cMet IC₅₀ that is at least 400 times greater than a Ron IC₅₀ of said compound.

In some cases, small changes in ring A or a substituent of ring A (A-R1) may modulate selectivity for Ron (e.g., over other protein kinases such as cMet). In some cases, small changes in ring C or a substituent of ring C may modulate inhibitory activity for Ron (e.g., affect a Ron IC₅₀). For example, a Ron inhibitor of Formula (I) with a low Ron IC₅₀ may be modified at Ring A (or a substituent thereof, A-R¹) to enhance its selectivity toward Ron.

The terms below, as used herein, have the following meanings, unless indicated otherwise:

“Amino” refers to the —NH2 radical.

“Cyano” refers to the —CN radical.

“Hydroxy” or “hydroxyl” refers to the —OH radical.

“Nitro” refers to the —NO2 radical.

“Oxo” refers to the ═O substituent.

“Aryl” may refer to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused or bridged ring systems. Aryl radicals include, but are not limited to, aryl radicals derived from the hydrocarbon ring systems of benzene, indane, indene, and naphthalene.

“Heteroaryl” may refer to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized.

“Alkyl” may refer to a straight or branched hydrocarbon chain radical, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl, an alkyl comprising up to 4 carbon atoms is a C₁-C₄ alkyl. Alkyls (and other moieties defined herein) comprising other numbers of carbon atoms are represented similarily. Representative alkyl groups include, but are not limited to, methyl, ethyl, n-propyl, 1-methylethyl (iso-propyl), n-butyl, i-butyl, s-butyl, n-pentyl, 1,1-dimethylethyl (t-butyl), 3-methylhexyl, 2-methylhexyl, and the like. Unless stated otherwise specifically in the specification, an alkyl group may be optionally substituted as described below. “Alkylene” or “alkylene chain” refers to a straight or branched divalent hydrocarbon chain linking the rest of the molecule to a radical group.

“Alkoxy” may refer to a radical of the formula —OR where R is an alkyl radical as defined. Unless stated otherwise specifically in the specification, an alkoxy group may be optionally substituted as described below.

“Heteroalkylene” may refer to an alkyl radical as described above where one or more carbon atoms of the alkyl is replaced with a O, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH2CH2OMe, OCH2CH2OCH2CH2NH2, or OCH2CH2OCH2CH2OCH2CH2N(Me)2.

“Cycloalkyl” may refer to a stable, non-aromatic, monocyclic or polycyclic carbocyclic ring, which may include fused or bridged ring systems, which is saturated or unsaturated, and attached to the rest of the molecule by a single bond. Representative cycloalkyls include, but are not limited to, cycloaklyls having from three to fifteen carbon atoms, from three to ten carbon atoms, from three to eight carbon atoms, from three to six carbon atoms, from three to five carbon atoms, or three to four carbon atoms. Monocyclic cyclcoalkyl radicals include, for example, cyclopropyl, cyclobutyl, cyclopentyl, cyclohexyl, cycloheptyl, and cyclooctyl. Polycyclic radicals include, for example, adamantyl, norbornyl, decalinyl, and 7,7-dimethyl-bicyclo[2.2.1]heptanyl.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” may refer to bromo, chloro, fluoro or iodo.

“Haloalkyl” may refer to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Heterocyclyl” or “heterocyclic ring” or “hetercycloalkyl” may refer to a stable 3- to 14-membered non-aromatic ring radical comprising 2 to 13 carbon atoms and from one to 6 heteroatoms selected from the group consisting of nitrogen, oxygen, and sulfur. Unless stated otherwise specifically in the specification, the heterocyclyl radical may be a monocyclic, or bicyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated.

### Delivery or Administration of One or More Agents

The present disclosure provides methods and compositions for delivery or administration of one or more agents described herein. One or more agents may be delivered to a subject (e.g., in vivo), or to a cell or population of cells from a subject (e.g., ex vivo or in vivo). In some cases, the one or more agents may be delivered to a subject in one or more delivery vesicles, such as a nanoparticle. The nanoparticle may be any suitable nanoparticle and may be a solid, semi-solid, semi-liquid or a gel. The nanoparticle may be a lipophilic and amphiphilic particle. For example, a nanoparticle may comprise a micelle, liposome, exosome, or other lipid-containing vesicle. In some cases, the nanoparticle may be configured for targeted delivery to a certain cell or cell type (e.g., cancer cell). In such cases, the nanoparticle may be decorated with any number of ligands, e.g., antibodies, nucleic acid molecules (e.g., ribonucleic acid (RNA) molecules or deoxyribonucleic acid (DNA) molecules), proteins, peptides, which may specifically bind to a certain cell or cell type (e.g., cancer cell).

The one or more agents may be delivered using viral approaches. For example, the one or more agents may be administered using a viral vector. In such cases, the one or more agents may be encapsulated in a virus for delivery to a cell, population of cells, or the subject. The virus can be an adeno-associated virus (AAV), a retrovirus, a lentivirus, a herpes simplex virus, or other useful virus. The virus may be engineered or may be naturally occurring.

The one or more agents may be delivered to a subject (e.g., human patient) or a body of the subject (e.g., at the tumor site) using a single or variety of approaches. For example, the one or more agents may be delivered or administered orally, intravenously, intraperitoneally, intratumorally, subcutaneously, topically, transdermally, transmucosally, or through another administration approach.

The one or more agents may be delivered to the subject enterally. For example, the one or more agents may be administered to the subject orally, rectally, sublingually, sub-labially, buccally, topically, or through an enema. In such cases, the one or more agents may be formulated into a tablet, capsule, drop or other formulation. The formulation may be configured to be delivered enterally.

The one or more agents may be delivered to the subject parenterally. For example, the one or more agents may be administered via injection into a location of the subject. The location may comprise the central nervous system, and the one or more agents may be delivered epidurally, intracerebrally, intracerebroventricularly, etc. The location may comprise the skin, and the one or more agents may be delivered epicutaneously. For instance, the one or more agents may be formulated in a transdermal patch, which can deliver the one or more agents to the skin of a subject. The one or more agents may be delivered sublingually and/or bucally, extra-amniotically, nasally, intra-arterially, intra-articularly, intravavernously, intracardiacally, intradermally, intralesionally, intramuscularly, intraocularly, intraosseously, intraperitoneally, intrathecally, intrauterinely, intravaginally, intravenously, intravesically, intravitreally, subcutaneously, trans-dermally, perivascularly, transmucosally, or through another route of administration. In some cases, the one or more agents may be delivered topically.

The one or more agents may be formulated into an aerosol, pill, tablet, capsule (e.g., asymmetric membrane capsule), pastille, elixir, emulsion, powder, solution, suspension, tincture, liquid, gel, dry powder, vapor, droplet, ointment, patch, or a combination thereof. For instance, the one or more agents may be formulated in a gel or polymer and delivered via a thin film.

In some instances, the one or more agents may be delivered to the subject using a targeted delivery approach (e.g., for targeted delivery to the tumor site) or using a delivery approach to increase uptake of a cell of the one or more agents. The delivery approach may comprise magnetic drug delivery (e.g., magnetic nanoparticle-based drug delivery), an acoustic targeted drug delivery approach, a self-microemulsifying drug delivery system, or other delivery approach. In some cases, the one or more agents may be formulated for targeted delivery or for increased uptake of a cell. For example, the one or more agents may be formulated with another agent, which may improve the solubility, hydrophobicity, hydrophilicity, absorbability, half-life, bioavailability, release profile, or other property of the one or more agents. For example, the one or more agents may be formulated with a polymer which may control the release profile of the one or more agents. The one or more agents may be formulated as a coating or with a coating (e.g., bovine submaxillary mucin coatings, polymer coatings, etc.) to alter a property of the one or more agents (e.g., bioavailability, pharmacokinetics, etc.).

In some instances, the one or more agents may be formulated using retrometabolic drug design. In such cases, the one or more agents may be assessed for metabolic effects in a cell, and a new formulation comprising a derivative (e.g., chemically synthesized alternative or engineered variant) may be designed to change a property of the one or more agents (e.g., to increase efficacy, minimize undesirable side effects, alter bioavailability, etc.).

### EXAMPLES

### Example 1—SRC-YES as a Synthetic Lethal Pair in Triple-Negative Breast Cancer

To determine whether a combination of targeted agents may be effective in killing triple-negative breast cancer, plausible synthetic lethal gene pair targets can first be identified using a genetic knockdown or knockout screen (e.g., using the workflow depicted in FIG. 1). The initial screen is performed by pairing agents that eliminate the activity of all possible pairs of tyrosine kinase genes. Each tyrosine kinase gene pair can then be designated a gene interaction score, based on whether the combination of the knockdown or knockout of both genes in the pair is statistically significantly more effective at cell killing than the numerically additive effect of the separate knockdown of the individual genes. CombiGEM technology (Wong A S, Choi G C, Cui C H, et al. “Multiplexed barcoded CRISPR-Cas9 screening enabled by CombiGEM.” Proceedings of the National Academy of Sciences of the United States of America 2016; 113:2544-90, incorporated by reference herein) can be used to validate the impact of individual or combinations of gene modulations through CRISPR/Cas9 gene manipulation. In some instances, CombiGEM technology can be used in a high-throughput format in one or more cell types to determine a set of genes (e.g., gene pairs, gene triplets, gene quadruplets, etc.) that may result in synthetic lethality. One-wise and two-wise, combinatorial genetic perturbation libraries targeting the tyrosine kinase genes may be used.

The gene interaction scores of each gene pair can then be calculated using the equation:

GI_{AB}=Z_{obs}−Zₑₓₚ=Z_{AB}−(Z_{A}−Z_{B})

where A refers to gene A (a first tyrosine kinase), B refers to gene B (a second tyrosine kinase), Z_{obs} is the observed phenotype, Zₑₓₚ is the expected phenotype, and Z_{AB} is the observed phenotype. In such cases, Z_{AB} is calculated as the averaged log-fold change of the count of double sgRNAs (i.e., for gene A and gene B knockout) at Day 20 relative to Day 0. Zₑₓₚ is the sum of Z_{A} and Z_{B}, and is defined as the average log-fold change of the count of sgRNAs targeting gene A and gene B, respectively, at Day 20 relative to Day 0. The gene interaction scores may then be ranked by magnitude of the modified gene interaction score. The ranked gene pairs are listed in Table 1.

The top-ranked gene pairs may be subjected to further screening to determine whether the gene pair may be synthetic lethal. For example, the SRC-YES gene pair may be tested for synthetic lethality using additional cell-lines and alternative methods of gene function knock-down (e.g., siRNA or small molecules). As in the screen, in one approach, SRC and YES genes may be knocked down or knocked out of a cell's genome using a combinatorial genetics CRISPR approach. In such an example, a DNA construct may be generated. The DNA construct may comprise a Src gRNA to direct an endonuclease (e.g., a Cas protein) to the SRC gene, as well as a Yes gRNA to direct an endonuclease (e.g., a Cas protein) to the YES gene. The Src gRNA and Yes gRNA may comprise a sequence homologous or complementary to a sequence on the endogenous SRC gene or YES gene, respectively. In some instances, the DNA construct may also comprise replacement genes to replace SRC and YES in the genome (e.g., dysfunctional sequences, random DNA sequences).

Control DNA constructs may also be generated. For example, to determine if the gene pair is synthetic lethal, it may be important to monitor the effect of disrupting each member of the gene pair, as well as the combination of the gene pair. Moreover, it may be important to monitor the effect of a negative control, in which a DNA construct comprising an ineffective gRNA e.g., non-specific gRNA as a “non-cutting” control may be constructed. In addition, to determine whether the order in which the replacement genes occur in the DNA construct influences the effect on the cells, a DNA construct comprising SRC-YES sequences may be compared to one that comprises YES-SRC sequences.

The DNA constructs may then be introduced to cancer cells (e.g., MDA-MB-231 cells) with an endonuclease, e.g., Cas9. The Cas9 may then replace, edit, or delete the SRC and YES genes in the treated cells, and in some cases, replace the SRC and YES genes in the genomes with the replacement genes in the DNA constructs. Proliferation or viability of the cells may then be monitored over time to determine the effectiveness of the treatment. The viability of the cells may be normalized or compared to a control population of cells that are not treated.

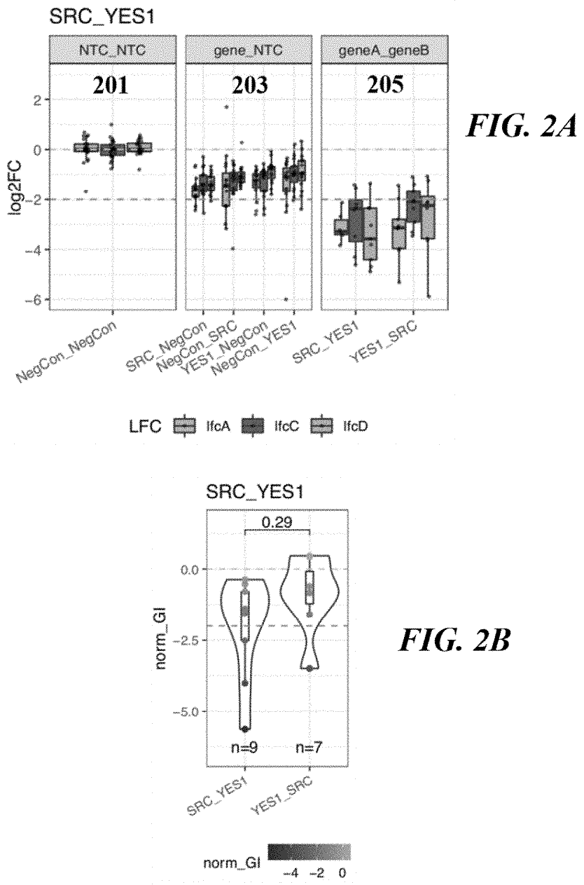

FIGS. 2A-2B show example data of a CombiGEM approach to knock out or knock down SRC and YES. FIG. 2A illustrates bar plots of cell viability as a function of the DNA construct introduced. FC represents fold-change over control. LFC represents log-fold change. A,B, C and D are biological replicates. In panel 201, MDA-MD-231 cells are treated with a negative control (NTC) DNA constructs which may comprise functional copies of SRC and YES, or may be otherwise configured to not affect (e.g., knock out) the SRC and YES genes in the cells. In panel 201, it can be noted that normalized cell viability is not affected by the negative control constructs. In panel 203, MDA-MD-231 cells are treated with a DNA construct configured to knock out only one member of the gene pair (SRC or YES). The DNA constructs tested can comprise: SRC-negative control, negative control-SRC, YES-negative control, and negative control-YES, which may also be used to determine whether the order in which the control and knockout sequences affects the cell viability. In panel 203, it can be noted that the viability of the cells can be decreased, indicating that YES knockdown or SRC knockdown can individually decrease cell viability. Moreover, the order in which the individual genes appear on the construct does not affect the effect of the single knockdown. In panel 205, MDA-MD-231 cells are treated with DNA constructs configured to knock out both SRC and YES. It can be noted that the viability of the cells is dramatically decreased compared to the single-gene knock down in 203 and the negative control in 201.

FIG. 2B illustrates a violin plot of viability of cells treated with DNA constructs to knock down or knock out SRC and YES. The DNA constructs can comprise SRC and YES gene replacements, such as to render the endogenous copies of SRC and YES non-functional. Knockdown of SRC and YES can result in decreased cell viability and demonstrate a substantial genetic interaction (GI<−1.5), and the difference in viability as a result of which order the genes appear on the DNA construct may not be significant (p=0.29).

To validate that the DNA constructs are correctly designed and that they indeed inhibit gene expression or activity of SRC and YES genes, a DNA analysis or a protein assay may be performed. For example, after introducing the DNA constructs to knock out or knock down SRC and YES genes in a cell, a Western Blot or other immunoassay may be used to ascertain that Src and Yes proteins are expressed at a lower level than a negative control population of cells.

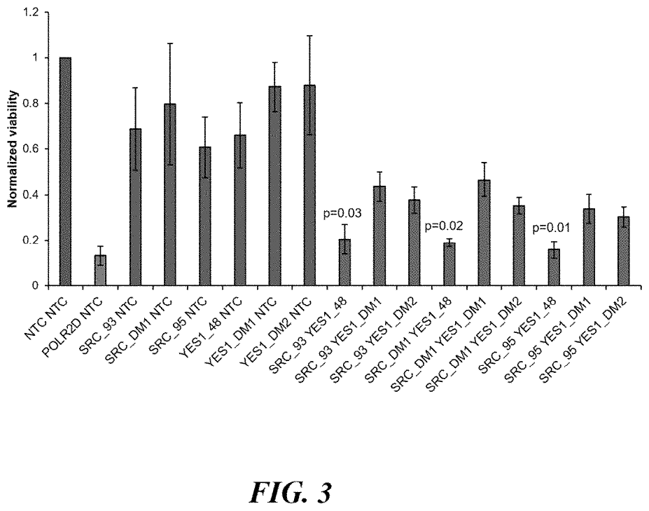

FIG. 3 shows additional example data of a CombiGEM approach to knock out or knock down SRC and YES in cancer (e.g., MBA-MD-231) cells. FIG. 3 shows bar plots of normalized viability of cells treated with a variety of DNA constructs comprising: (i) a negative control (NTC) sequence, (ii) a polymerase (POLR2D) sequence as a positive control for an essential gene, (iii) SRC guide RNA (for knock down or knockout), (iv) a YES guide RNA (for knock down or knockout), or (v) a combination thereof. The positive control sequence can be a DNA construct comprising a dysfunctional RNA polymerase gene (e.g., POLR2D gene) to replace the endogenous POLR2D gene, or the DNA construct may be configured to knock down or knock out a polymerase gene. The positive control sequence may be used, for example, to determine that the DNA constructs function as expected, e.g., knock out of a gene essential for DNA replication, and thus cell proliferation, results in decreased cell viability.

The viability of the treated cells can be normalized to a negative control (e.g., non-treated cells, or cells treated with DNA constructs comprising scrambled gRNA or comprising normal copies of SRC and YES). As can be seen in FIG. 3, the negative control group of cells has a normalized viability of 1. The positive control (POLR2D), where the cells are treated with a DNA construct to knockout a polymerase, results in dramatically decreased normalized viability, as expected. In bars 3-8 from the left, the cells are treated with a single gene knockout, either SRC (bars 3-5) or YES (bars 6-8). The terms “DM1” and “DM2” are used for nomenclature purposes. Knock out of SRC or YES results in decreased normalized viability. In bars 9-17 from the left, the cells are treated with a double gene knockout (SRC and YES), resulting in much lower viability than the single-knockout and negative control. Error bars represent standard deviation, n=3.

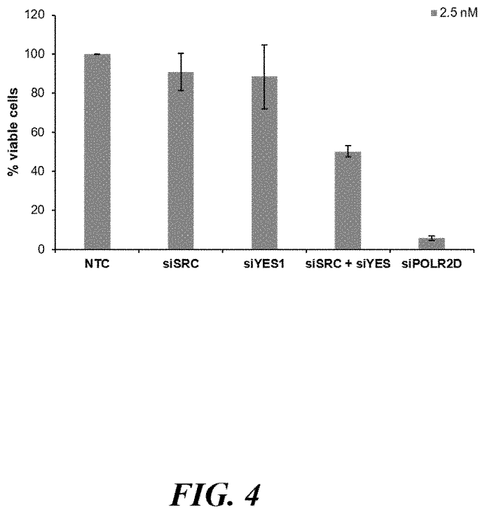

FIG. 4 shows additional example data of an RNA approach to knock down SRC and YES in cancer cells. Using an orthogonal approach to reduce expression or activity of SRC and YES, the results of the CombiGEM approach (e.g., FIG. 3) can be validated. Cells (e.g., MBA-MD-231 cells) may be treated with siRNA to knock down SRC, YES, or both. The concentration of siRNA used can be any suitable concentration (e.g., 2.5 nM). Following treatment, cells may be cultured for an additional duration of time (e.g., a week). Cell viability may then be compared or normalized to a negative control (e.g., cells treated with scrambled siRNA). FIG. 4 shows a bar plot of cell viability as a function of treatment group (negative control or no treatment (e.g., no RNA treatment, scrambed siRNA treatment), siSRC treatment, siYES treatment, or siPOLR2D). Cells treated with siSRC (to inhibit SRC) and cells treated with siYES (to inhibit YES) do not have a significantly decreased viability. However, cells treated with both siSRC and siYES have a significantly lower viability and illustrate a synergistic effect on decreasing viability compared to siSRC or siYES alone. The positive control is a siPOLR2D (RNA polymerase II knock down).

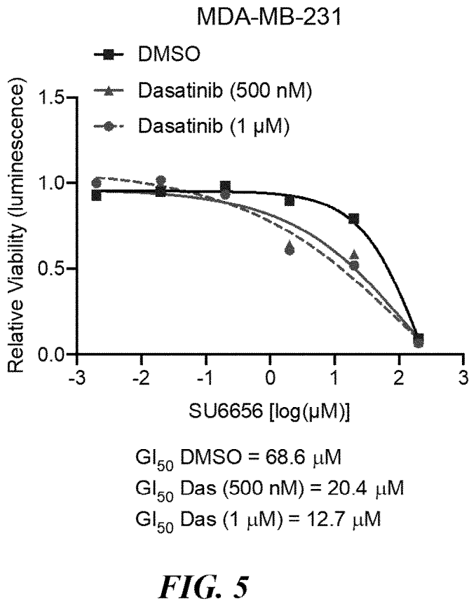

FIG. 5 shows additional example data of a small molecule approach to inhibit SRC and YES in cancer cells. An additional approach to validate the CombiGEM results (dual SRC and YES knockout using Cas and DNA constructs) can include using small molecule inhibitors to reduce the function of SRC or YES. FIG. 5 shows a plot of MDA-MB-231 cell viability as a function of concentration of SU-6656 (a YES inhibitor). One group of cells is treated with only the YES inhibitor and the vehicle control (DMSO only). Two groups of cells are co-treated with Dasatinib, a SRC inhibitor, at a concentration of 500 nM or 1 μM. The control group (DMSO only, no Dasatinib) has a 50% viability at a concentration of 68.6 μM of SU-6656, compared to 20.4 μM for the cells co-treated with 500 nM Dasatinib and 12.7 μM for the cells co-treated with 1 μM Dasatinib. These results indicate that treatment with a Yes inhibitor sensitizes cells to a SRC inhibitor. These results can suggest that dual inhibition of SRC and YES can be lethal to cells, compared to single inhibition or SRC or YES.

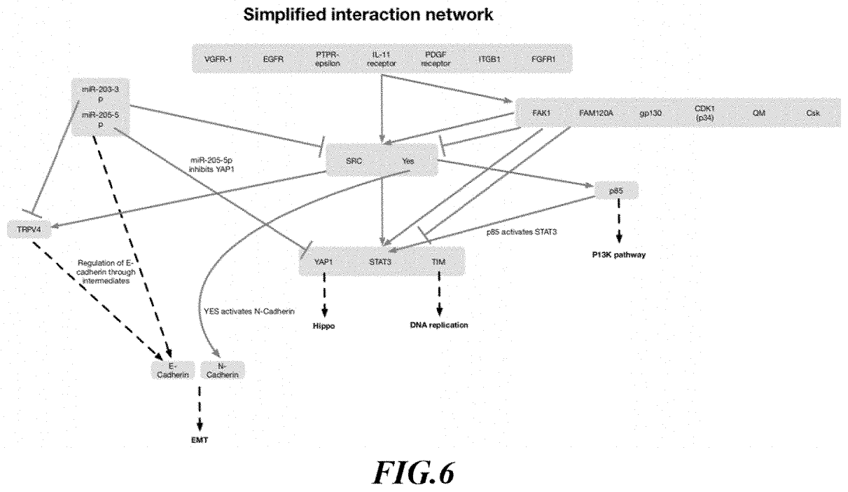

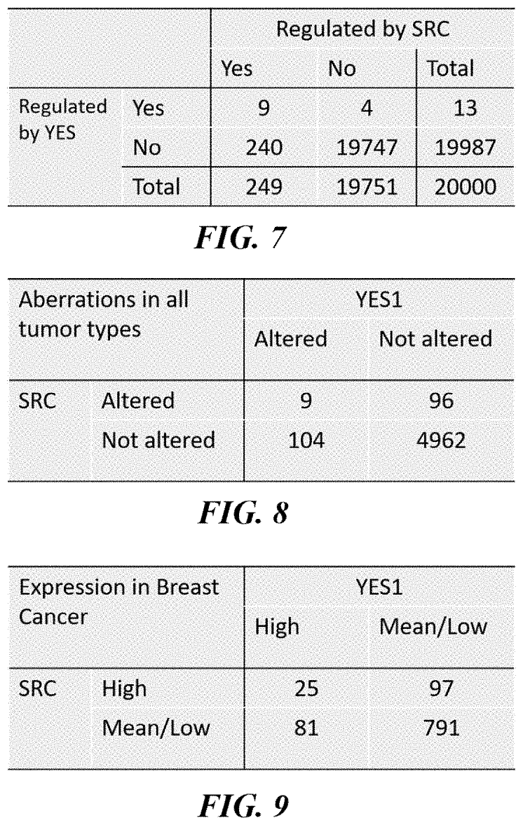

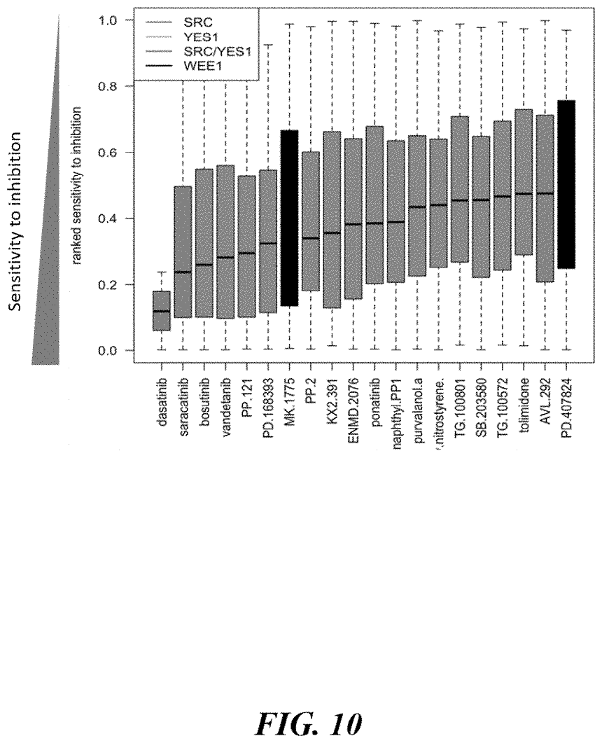

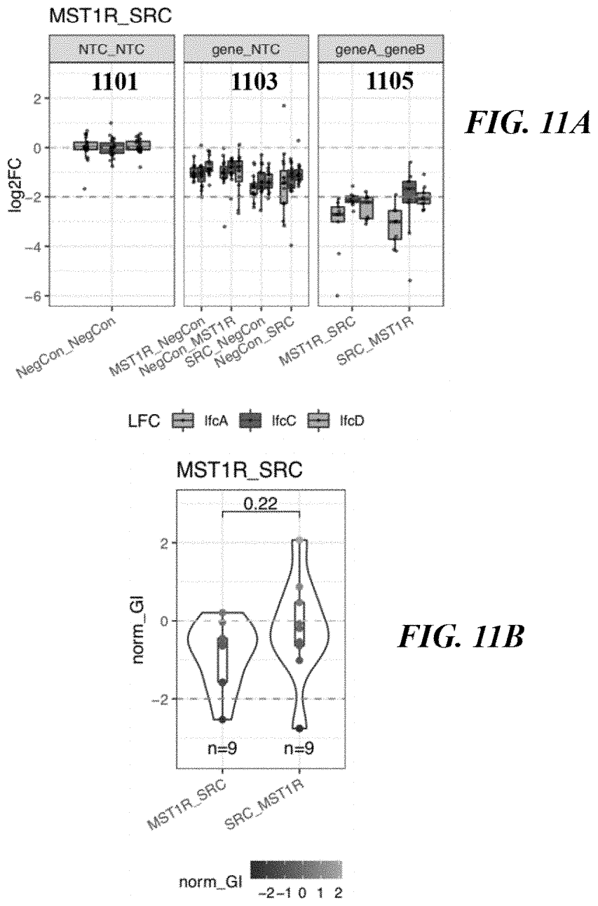

FIG. 6 schematically illustrates a subset of signaling pathways for YES and SRC. As can be seen, there is significant convergence between downstream genes and/or proteins regulated by YES and SRC. For example, YES and SRC can activate STAT3, TIM (involved in DNA replication), and YAP1 (involved in the Hippo pathway). YES and SRC may be regulated by a number of receptors, e.g., VGFR-1, EGFR, PTPR-epsilon, IL-11 receptor, PDGF receptor, ITGB1, and FGFR1. These receptors may also regulate a number of genes and/or proteins e.g., FAK1, FAM120Am gp130, and CDK1 (p34). Some of these may in turn activate expression or activity of SRC and YES, while some of these may in turn inhibit expression or activity of SRC and YES. Furthermore, some of these may in turn activate expression of activity of YAP1, STAT3 and TIM, while some may in turn inhibit expression or activity of YAP1, STAT3 and TIM. YES may also be involved in cadherin-induced signaling and the PI3k pathway (e.g., via activation of p85). SRC and YES may be involved in TRPV4 signaling, which can also regulate cadherin signaling. The micro-RNAs miR-203-3p and miR-205-5p may both inhibit expression of SRC and YES. MiR-203-2p may also inhibit TRPV4 expression and miR-205-5p may inhibit expression of YAP1. Both miR-203-3p and miR205-5p may be involved in cadherin-induced signaling. FIG. 7 shows a table of a number of genes that are regulated by YES or by SRC. As indicated in FIG. 7, of the 13 genes regulated by YES1, 9 of those genes are also regulated by SRC. YES and SRC are also important regulators in multiple cancer-related processes. FIG. 8 illustrates a table of instances of aberrations, e.g., mutations or deletions, of SRC or YES1 in different tumor types. SRC and YES1 are statistically significantly more likely to be co-mutated in the same tumor cell (p<0.001). FIG. 9 illustrates a table of expression of SRC and YES1 in breast cancer. SRC and YES1 are more likely to be highly co-expressed in breast cancer tumors (p<0.001).

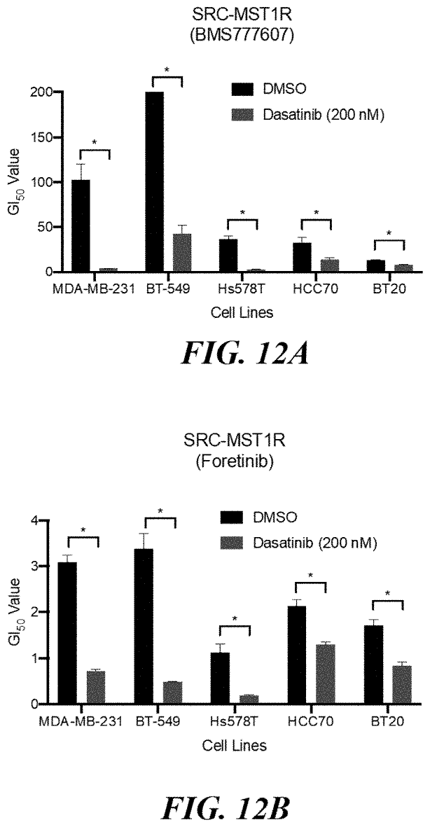

FIG. 10 shows example data of sensitivity of cells to YES and SRC inhibitors. The bar plot indicates the effect of various SRC inhibitors, YES inhibitors, or dual SRC/YES inhibitors on the sensitivity of the cells to inhibition; along with an inhibitor for an unrelated/control kinase WEE1. Each bar represents a different type of inhibitor, which may inhibit SRC, YES, SRC and YES, or WEE1. Bars 1-2 from the left of the plot indicate cells are most sensitive to inhibition when SRC and YES are both inhibited. Bars 3-6 from the left of the plot indicate sensitivity to inhibition for cells treated with SRC inhibitors. Bar 7 from the left of the plot indicates sensitivity to inhibition for cells treated with WEE1 inhibitor. Bars 8-18 from the left of the plot also indicate sensitivity to inhibition for cells treated with SRC inhibitors. Bar 9 from the left of the plot indicates sensitivity to inhibition for cells treated with YES1 inhibitor. Bar 10 from the left of the plot indicates sensitivity to inhibition for cells treated with WEE1 inhibitor. Increased sensitivity to inhibition is observed in SRC and YES dual inhibition, compared to SRC-only inhibition or YES-only inhibition. In FIG. 10, 506 cell lines are compared for sensitivity to Dasatinib (SRC and YES inhibitor). 119 of the 506 cell lines are more sensitive to Dasatinib than MDA-MB-231 cells. Cell lines that are sensitive to Dasatinib are generally less sensitive to more selective YES inhibitors or SRC inhibitors. Cell lines that are sensitive to Dasatinib are more sensitive to other dual inhibitors of YES and SRC.

Altogether, the data represented in FIGS. 2-10 suggest that SRC-YES may be a synthetic lethal pair. As SRC and YES are involved in similar cancer pathways, SRC and YES may have redundant functions in oncolytic processes. Moreover, SRC and YES are commonly, in tumor cells, more often co-expressed and mutated. In sensitive cells, dual SRC-YES inhibition can be more effective at killing cells than YES-only or SRC-only inhibition. Thus, dual specificity inhibitors, or combinations of mono-specific (e.g., YES-only or SRC-only) agents may be particularly effective in killing cancer cells.

### Example 2—SRC-RON as a Synthetic Lethal Pair in Triple-Negative Breast Cancer

As described in Example 1, the top-ranked gene pairs from a gene interaction screen may be subjected to further screening to determine whether the gene pair may be synthetic lethal. For example, the SRC-RON (where RON is also called MST1R) gene pair may be tested for synthetic lethality. In one approach, SRC and RON genes may be knocked down or knocked out of a cell's genome using a CRISPR-based approach. In such an example, a DNA construct may be generated. The DNA construct may comprise a Src gRNA to direct an endonuclease (e.g., a Cas protein) to the SRC gene, as well as a RON gRNA to direct an endonuclease (e.g., a Cas protein) to the RON gene. The Src gRNA and RON gRNA may comprise a sequence homologous or complementary to a sequence on the endogenous SRC gene or RON gene, respectively. In some instances, the DNA construct may also comprise replacement genes to replace SRC and RON in the genome (e.g., dysfunctional sequences, random DNA sequences).

Control DNA constructs may also be generated. For example, to determine if the gene pair is synthetic lethal, it may be important to monitor the effect of disrupting each member of the gene pair, as well as the combination of the gene pair. Moreover, it may be important to monitor the effect of a negative control, in which a DNA construct comprising an ineffective gRNA (e.g., non-specific gRNA as a “non-cutting” control), normal copies of the SRC and RON genes (to affect minimal change), or a combination thereof may be constructed. In addition, to determine whether the order in which the replacement genes occur in the DNA construct influences the effect on the cells, a DNA construct comprising SRC-RON sequences may be compared to one that comprises RON-SRC sequences.

The DNA constructs may then be introduced to cancer cells (e.g., MDA-MB-231 cells) with an endonuclease, e.g., Cas9. The Cas9 may then replace, edit, or delete the SRC and RON genes in the treated cells, and in some cases, replace the SRC and RON genes in the genomes with the replacement genes in the DNA constructs. Proliferation or viability of the cells may then be monitored over time to determine the effectiveness of the treatment. The viability of the cells may be normalized or compared to a control population of cells e.g., cells that are not treated or cells treated with a scrambled gRNA, or cells treated with only a vehicle control.

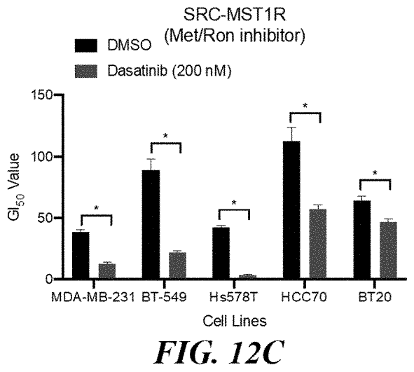

FIGS. 11A-11B show example data of a CombiGEM approach to knock out or knock down SRC and RON (illustrated as “MST1R”). FIG. 11A illustrates bar plots of cell viability as a function of the DNA construct introduced. FC represents fold-change over control. LFC represents log-fold change. A,B, C and D are biological replicates. In panel 1101, MDA-MD-231 cells are treated with a negative control (NegCon) DNA constructs which may comprise functional copies of SRC and RON, or may be otherwise configured to not affect (e.g., knock out) the SRC and RON genes in the cells. In panel 1101, it can be noted that normalized cell viability is not affected by the negative control constructs. In panel 1103, MDA-MD-231 cells are treated with a DNA construct configured to knock out only one member of the gene pair (SRC or RON). The DNA constructs tested can comprise: SRC-negative control, negative control-SRC, RON-negative control, and negative control-RON, which may also be used to determine whether the order in which the control and knockout sequences affects the cell viability. In panel 1103, it can be noted that the viability of the cells can be decreased, indicating that RON knockdown or SRC knockdown can individually decrease cell viability. Moreover, the order in which the individual genes appear on the construct does not affect the effect of the single knockdown on cell viability. In panel 1105, MDA-MD-231 cells are treated with DNA constructs configured to knock out both SRC and RON. It can be noted that the viability of the cells is dramatically decreased compared to the single-gene knock down in 1103 and the negative control in 1101.

FIG. 11B illustrates a violin plot of viability of cells treated with DNA constructs to knock down or knock out SRC and RON. The DNA constructs can comprise SRC and RON gene replacements, such as to render the endogenous copies of SRC and RON non-functional. Knockdown of SRC and RON can result in decreased cell viability, and the difference in viability as a result of which order the genes appear on the DNA construct may not be significant (p=0.22).

To validate that the DNA constructs are correctly designed and that they indeed inhibit gene expression or activity of SRC and RON genes, a protein assay may be performed. For example, after introducing the DNA constructs to knock out or knock down SRC and RON genes in a cell, a Western Blot or other immunoassay may be used to ascertain that Src and RON proteins are expressed at a lower level than a negative control population of cells.

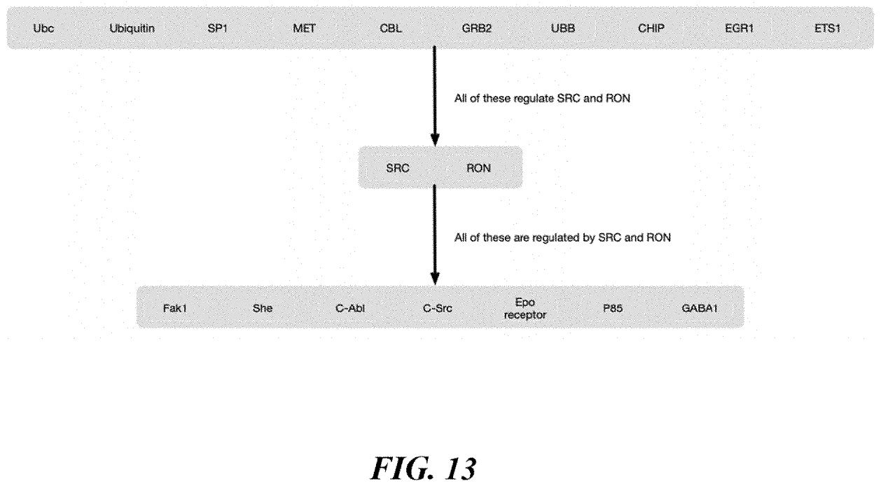

FIGS. 12A-12C show additional example data of a small molecule approach to inhibit SRC and RON in cancer cells. As described herein, one approach to validate the CombiGEM results (dual SRC and RON knockout using Cas and DNA constructs) can include using small molecule inhibitors to reduce function of SRC or RON. FIG. 12A shows a plot of cellular IC₅₀ (GI₅₀) as a function of treatment with N-[4-[(2-amino-3-chloro-4-pyridinyl)oxy]-3-fluorophenyl]-4-ethoxy-1-(4-fluorophenyl)-2-oxo-3-pyridinecarboxamide (BMS777607, a RON inhibitor), or when BMS777607 is co-administered with Dasatinib (a SRC inhibitor). Five cell line types are displayed. For all tested cell lines, the potency (lower GI50) is greater for the RON inhibitor in the presence of Dasatinib than in its absence (DMSO). FIG. 12B and FIG. 12C show plots of GI₅₀ as a function of treatment with RON inhibitors Foretinib or a Met/Ron inhibitor, respectively, co-administered with Dasatinib. In all tested cell lines, the potency (lower GI50) is greater for the RON inhibitor in the presence of Dasatinib than in its absence (DMSO). Altogether, these results suggest that treatment with a RON inhibitor can sensitive cells to a SRC inhibitor. These results can also suggest that dual inhibition of SRC and RON can be lethal to cells, compared to single inhibition or SRC or RON. It also demonstrates that the effect is not limited to only one cell type—that is there is good penetrance of this effect.

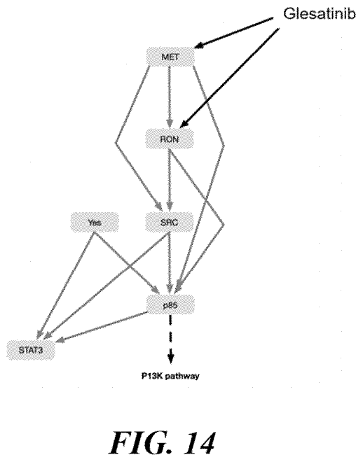

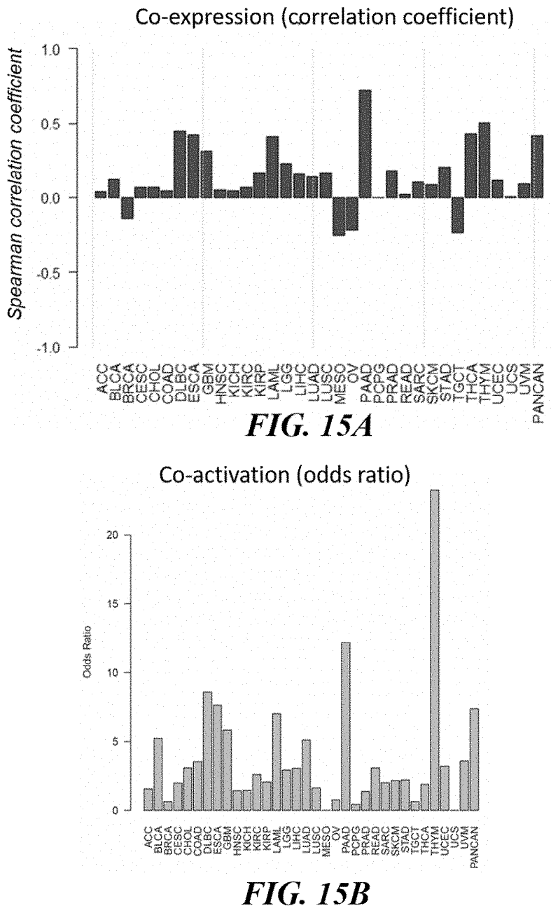

FIG. 13 schematically illustrates a subset of signaling pathways for RON (MST1R) and SRC. As can be seen, there is convergence between downstream genes regulated by RON and SRC. Both genes are expressed in most cancer samples, and multiple genes are regulated by SRC and RON. FIG. 14 schematically shows a subset of the signaling pathways forp85 (PI3K pathway), indicating that RON and SRC interact. MET and RON, upstream of SRC, may be inhibited using Glesatinib.

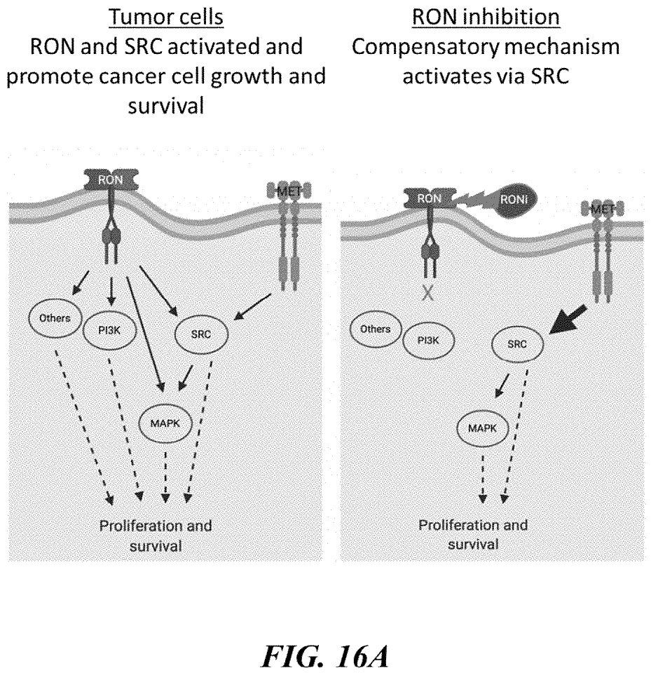

FIG. 15A shows a plot of correlation coefficients of the expression of SRC and RON in various cancer types. Each bar in the plot represents a Spearman correlation coefficient of expression of SRC and RON in a cancer type. SRC and RON are highly co-expressed in many cancer types. FIG. 15B shows a plot of co-activation of SRC and RON in various cancer types. Each bar in the plot represents the odds ratio of a cancer being activated for SRC given it is activated for RON. SRC and RON are significantly co-activated in multiple cancer types.

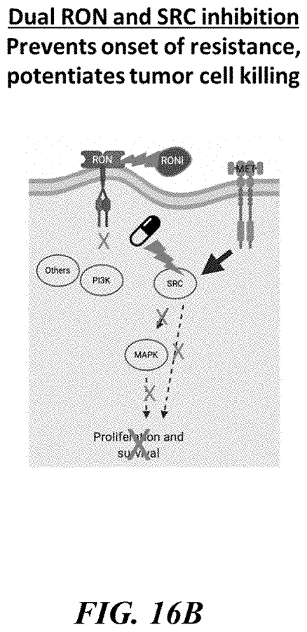

FIG. 16A schematically shows a subset of the signaling pathways for SRC and RON. In some tumor cells, RON and SRC are activated and promote cancer cell growth and survival. Inhibition of RON may block parts of the pathway (e.g., P13K). However, a compensatory mechanism may be active via SRC that continues to promote cancer cell growth and survival. FIG. 16B schematically shows how dual inhibition of SRC and RON may be effective in suppressing tumor growth and possibly potentiating tumor cell death.

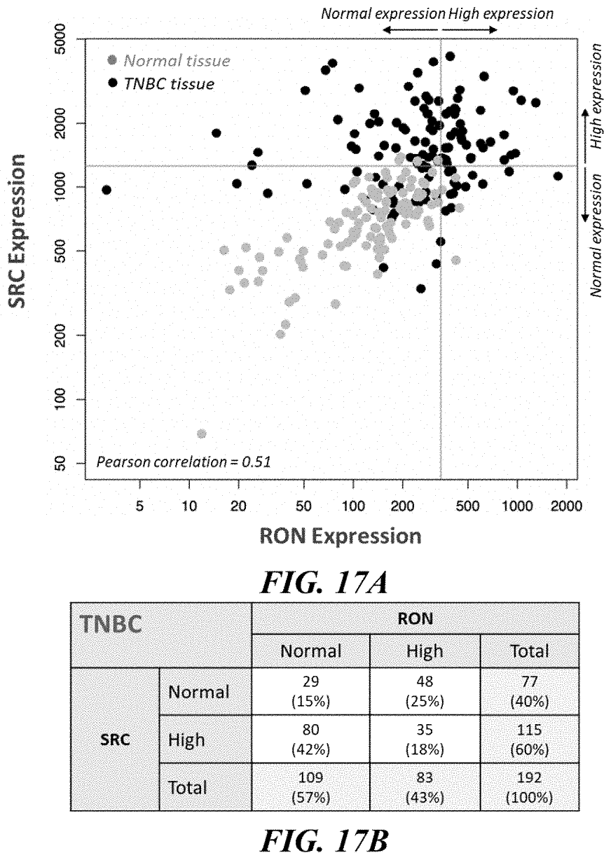

FIG. 17A shows a plot of RON expression versus SRC expression in normal and cancerous tissues. The gray dots represent expression levels of RON and SRC of normal tissue cells and the black dots represent expression levels of RON and SRC in triple-negative breast cancer cells. FIG. 17B shows a table of expression levels of SRC and RON in triple-negative breast cancer cells. “High” corresponds to expression levels that are at least two standard deviations above the mean expression levels of normal tissues. As illustrated in FIGS. 17A-B, a positive correlation of SRC and RON expression in tumor cells can be observed, suggesting potential for dual inhibition. Further, 85% of triple-negative breast cancer samples (n=192) have high expression of either SRC and RON, and 18% have high expression of both SRC and RON.

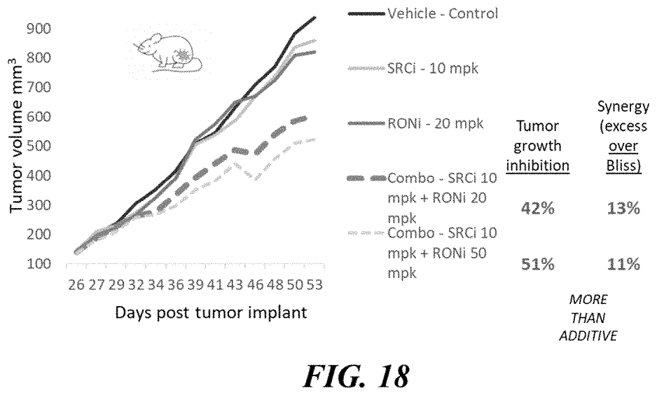

FIG. 18 shows example in vivo data of tumor volume as a function of treatment with SRC or RON inhibitor. MDA-MB-231 tumor xenografts are implanted with mice and treated with one of five experimental groups: vehicle (negative) control, SRC inhibitor (Dasatinib) at a concentration of 10 milligrams per kilogram (mpk), RON inhibitor (BMS777607) at 20 mpk, a combination of SRC inhibitor (10 mpk) and RON inhibitor (20 mpk), or a combination of SRC inhibitor (10 mpk) and RON inhibitor (50 mpk). The tumor volumes for each mouse are monitored over time. As can be observed, tumor growth in SRC-only inhibited or RON-only inhibited groups is slightly lower compared to the vehicle control, whereas the combination treatments of SRC inhibitor and RON inhibitors (either 20 mpk or 50 mpk) inhibit tumor growth by 42% and 51%, respectively. The calculated synergy of the dual inhibition of SRC and RON, compared to the addition of single inhibition (SRC-only and RON-only) is 13% and 11% for the 20 mpk and 50 mpk RON inhibitor groups, respectively.

Altogether, the data represented in FIGS. 11-18 suggest that SRC-RON may be a synthetic lethal pair and that dual inhibition of SRC and RON may be an effective treatment for cancer. SRC and RON are commonly, in tumor cells, co-expressed and activated. In sensitive cells, dual SRC-RON inhibition can be more effective at killing cells than RON-only or SRC-only inhibition. Thus, dual specificity inhibitors, or combinations of mono-specific (e.g., RON-only or SRC-only) agents may be particularly effective in killing cancer cells.

### Example 3—Improved Compounds for Selectively Inhibiting RON

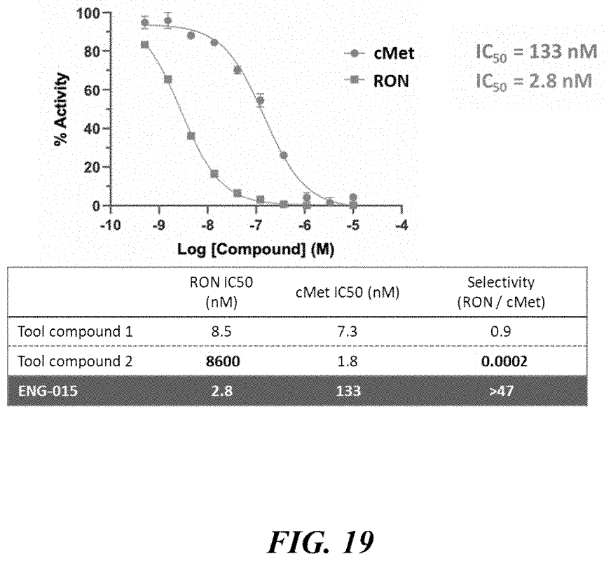

Improvements to selectivity, specificity, or potency of RON and/or SRC inhibitors may also improve cancer treatment, e.g., by requiring lower effective dosages or decreasing off-target effects. FIG. 19 shows example data of a compound (“ENG-015”) that has higher potency and selectivity in RON inhibition than tool compound 1 (ENG-007, BMA777607, N-(4-((2-amino-3-chloropyridin-4-yl)oxy)-3-fluorophenyl)-4-ethoxy-1-(4-fluorophenyl)-2-oxo-1,2-dihydropyridine-3-carboxamide) and tool compound 2 (ENG-008, 4-(3-(2-isopropoxyethoxy)-1H-indazol-5-yl)-2,6-dimethyl-1,4-dihydropyridine-3,5-dicarbonitrile). The plot indicates percent activity of RON or cMet as a function of the log concentration of the compound (in moles, M). From the plot, the IC₅₀ values may be obtained. For example, for ENG-015, the IC₅₀ is 2.8 nM for RON and 133 nM for cMet. In comparison, the IC₅₀ values for RON for tool compounds 1 and 2 are 8.5 and 8600, respectively, and the IC₅₀ values for cMET for tool compounds 1 and 2 are 7.3 and 1.8, respectively. The selectivity of tool compounds 1 and 2 for RON over cMet, as measured by cMet IC₅₀/RON IC₅₀, are 0.9 and 0.0002, respectively. In contrast, ENG-015 has a selectivity for RON over cMet that is >47, indicating that ENG-015 is a much more selective compound for inhibiting RON (with lower off-target cMet effects) than either tool compounds 1 and 2. The chemical composition of ENG-015 is illustrated in FIG. 20.

FIG. 20 shows the chemical compositions of a plurality of compounds that inhibit RON, some of which may have minimal or reduced off-target effects (e.g., reduced effect on cMet). The four compounds “ENG-009”, “ENG-015”, “ENG-018”, and“ENG-035” are shown, along with their Ron and cMet inhibitory activities. Some of the compounds of FIG. 20 may exhibit greater selectivity or potency for inhibiting RON compared to the conventional RON inhibitors shown in FIG. 21. For example, ENG-015 has an IC₃₀ for cMet of 133-142 nM and IC₃₀ for RON of 2.8 nM, indicating that using a therapeutically effect amount of ENG-015 can inhibit RON without inhibiting cMet. The selectivity (as determined by cMet IC₅₀/RON IC₅₀) of ENG-015 for RON over cMet is approximately 50-fold and ENG-015 is highly potent (requires a relatively low concentration to effectively inhibit RON, RON IC₅₀ of 2.8 nM). ENG-009 and ENG-035 exhibit lower potency (RON IC₅₀ of 222-291 nM and 43 nM dissociation constant (K_{d}), respectively) but are inactive for cMet (ENG-009) or ˜2.3-fold more selective for RON over cMet (ENG-035). ENG-018 is relatively inactive for both RON and cMET inhibition.

FIG. 21 shows the chemical compositions of a plurality of known compounds that inhibit RON, some of which exhibit increased potential off-target effects (e.g., cross-reactivity or inhibition of cMet). The IC₃₀ for cMet is considerably lower for several of the compounds, indicating that RON inhibition may likely yield cMet inhibition as well. For example, ENG-014 is not more selective for RON than cMet. In contrast, ENG-013 is approximately 10-fold more selective for RON than cMet (as determined by cMet IC₅₀/RON IC₅₀) and potent (low RON IC₅₀ of 1.6 nM). ENG-012 is relatively potent (low RON IC₅₀ value of 3.3 nM) and is relatively selective (approximately 3-fold more selective for RON than cMet).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-58. (canceled)

59. A method for treating a subject having or suspected of having a cancer, comprising administering to said subject therapeutically effective amounts of one or more agents that cause a decrease in expression or activity of both members of one or more gene pairs selected from Table 1.

60. The method of claim 59, wherein said one or more agents comprise one or more members selected from the group consisting of a small molecule, a protein, a peptide, a ribonucleic acid (RNA) molecule, and, an endonuclease complex and a deoxyribonucleic acid (DNA) construct.

61. The method of claim 60, wherein said one or more agents comprise said DNA construct, and wherein said DNA construct comprises an endonuclease gene.

62. The method of claim 61, wherein said endonuclease gene encodes a CRISPR associated (Cas) protein.

63. The method of claim 62, wherein said Cas is Cas9.

64. The method of claim 62, wherein said DNA construct comprises a guide RNA directed to a gene of said one or more gene pairs.

65. The method of claim 60, wherein said one or more agents comprise said endonuclease complex, and wherein said endonuclease comprises a Cas protein.

66. The method of claim 60, wherein said one or more agents comprise said small molecule, and wherein said small molecule comprises a Src inhibitor.

67. The method of claim 66, wherein said Src inhibitor comprises N-(2-chloro-6-methylphenyl)-2-[[6-[4-(2-hydroxyethyl)-1-piperazinyl]-2-methyl-4-pyrimidinyl]amino]-5-thiazolecarboxamide monohydrate (Dasatinib), N-(5-chloro-1,3-benzodioxol-4-yl)-7-(2-(4-methylpiperazin-1-yl)ethoxy)-5-(tetrahydro-2H-pyran-4-yloxy)quinazolin-4-amine (Saracatinib), 4-[(2,4-dichloro-5-methoxyphenyl)amino]-6-methoxy-7-[3-(4-methylpiperazin-1-yl)propoxy]quinoline-3-carbonitrile (bosutinib), (4-Amino-5-(4-methylphenyl)-7-(t-butyl)pyrazolo[3,4-d]-pyrimidine), (4-chlorophenyl)-7-(t-butyl)pyrazolo[3,4-d]-pyrimidine) (PP1), 1-tert-butyl-3-(4-chlorophenyl)pyrazolo[3,4-d]pyrimidin-4-amine (PP2), 6-(2,6-dichlorophenyl)-2-{[3-(hydroxymethyl)phenyl]amino}-8-methyl-7H,8H-pyrido[2,3-d]pyrimidin-7-one (PD1663266), (E)-N-[4-[3-chloro-4-(pyridin-2-ylmethoxy)anilino]-3-cyano-7-ethoxyquinolin-6-yl]-4-(dimethylamino)but-2-enamide (Neratinib), 3-(2-imidazo[1,2-b]pyridazin-3-ylethynyl)-4-methyl-N-[4-[(4-methylpiperazin-1-yl)methyl]-3-(trifluoromethyl)phenyl]benzamide (Ponatinib), (E)-N-[4-(3-chloro-4-fluoroanilino)-3-cyano-7-ethoxyquinolin-6-yl]-4-(dimethylamino)but-2-enamide (Pelitinib), N-benzyl-2-[5-[4-(2-morpholin-4-ylethoxy)phenyl]pyridin-2-yl]acetamide (Tirbanibulin), 4-methyl-3-[(2-methyl-6-pyridin-3-ylpyrazolo[3,4-d]pyrimidin-4-yl)amino]-N-[3-(trifluoromethyl)phenyl]benzamide (NVP-BHG712), (2S,3S)-2,3-dihydroxybutanedioic acid; 6-(4-methylpiperazin-1-yl)-N-(5-methyl-1H-pyrazol-3-yl)-2-[(E)-2-phenylethenyl]pyrimidin-4-amine (ENMD-2076), 4-[4-[(5-tert-butyl-2-quinolin-6-ylpyrazol-3-yl)carbamoylamino]-3-fluorophenoxy]-N-methylpyridine-2-carboxamide (Rebastinib), or any combination thereof.

68. The method of claim 66, wherein said Src inhibitor comprises N-(2-chloro-6-methylphenyl)-2-[[6-[4-(2-hydroxyethyl)-1-piperazinyl]-2-methyl-4-pyrimidinyl]amino]-5-thiazole carboxamide monohydrate (Dasatinib).

69. The method of claim 60, wherein said one or more agents comprise said small molecule, and wherein said small molecule comprises a Yes inhibitor.

70. The method of claim 69, wherein said Yes inhibitor comprises (3Z)-N,N-Dimethyl-2-oxo-3-(4,5,6,7-tetrahydro-1H-indol-2-ylmethylidene)-2,3-dihydro-1H-indole-5-sulfonamide) (SU-6656).

71. The method of claim 60, wherein said one or more agents comprise said small molecule, and wherein said small molecule comprises a Ron inhibitor.

72. The method of claim 71, wherein said Ron inhibitor comprises N-[4-[(2-amino-3-chloro-4-pyridinyl)oxy]-3-fluorophenyl]-4-ethoxy-1-(4-fluorophenyl)-2-oxo-3-pyridinecarboxamide (BMS77607), N-(3-fluoro-4-((2-(1-methyl-1H-imidazol-4-yl)thiazolo[5,4-d]pyrimidin-7-yl)oxy)phenyl)-1-phenyl-5-(trifluoromethyl)-1H-pyrazole-4-carboxamide (ENG-009), N-(3-fluoro-4-((7-(1-methyl-1H-imidazol-4-yl)-1,6-naphthyridin-4-yl)oxy)phenyl)-1-phenyl-5-(trifluoromethyl)-1H-pyrazole-4-carboxamide (ENG-015), N-(3-fluoro-4-((6-(1-methyl-1H-imidazol-4-yl)-1H-indazol-3-yl)oxy)phenyl)-1-phenyl-5-(trifluoromethyl)-1H-pyrazole-4-carboxamide (ENG-018), N-(3-fluoro-4-((2-(1-methyl-1H-imidazol-4-yl)thiazolo[4,5-b]pyridin-7-yl)oxy)phenyl)-1-phenyl-5-(trifluoromethyl)-1H-pyrazole-4-carboxamide (ENG-035), N-(3-fluoro-4-((2-(1-methyl-1H-imidazol-4-yl)thieno[3,2-b]pyridin-7-yl)oxy)phenyl)-1-phenyl-5-(trifluoromethyl)-1H-pyrazole-4-carboxamide (ENG-006), N1′-[3-fluoro-4-[[6-methoxy-7-[3-(4-morpholinyl)propoxy]-4-quinolinyl]oxy]phenyl]-N1-(4-fluorophenyl)cyclopropane-1,1-dicarboxamide (foretinib), 1-(2-(2,6-difluorophenyl)-4-oxothiazolidin-3-yl)-3-(4-((7-(3-(4-ethylpiperazin-1-yl)propoxy)-6-methoxyquinolin-4-yl)oxy)-3,5-difluorophenyl)urea (ENG-013), N-[4-[(2-amino-3-chloro-4-pyridinyl)oxy]-3-fluorophenyl]-4-ethoxy-1-(4-fluorophenyl)-2-oxo-3-pyridinecarboxamide (ENG-007), or any combination thereof.

73. The method of claim 72, wherein said Ron inhibitor comprises N-[4-[(2-amino-3-chloro-4-pyridinyl)oxy]-3-fluorophenyl]-4-ethoxy-1-(4-fluorophenyl)-2-oxo-3-pyridinecarboxamide (BMS777607) or N1′-[3-fluoro-4-[[6-methoxy-7-(3-morpholinopropoxy)-4-quinolyl]oxy]phenyl]-N1-(4-fluorophenyl)cyclopropane-1,1-dicarboxamide (Foretinib).

74. The method of claim 59, wherein said cancer is breast cancer.

75. The method of claim 74, wherein said breast cancer is triple-negative breast cancer.

76. The method of claim 59, wherein said gene pair comprises Ron and Src.

77. The method of claim 76, wherein said Src comprises a constitutively active Src.

78. The method of claim 76, wherein said Ron comprises a short-form Ron.
